(19) United States
(12) Patent Application Publication
(10) Pub. No.: US 2026/0165593 A1
(43) Pub. Date: Jun. 18, 2026

(54) THIN-FILM DIAPHRAGM CAPACITIVE ELECTRODES

(71) Applicant: Edwards Lifesciences Corporation, Irvine, CA (US)

(72) Inventors: Gustavo Rios, Pasadena, CA (US); Alexander H. Siemons, Yorba Linda, CA (US)

(21) Appl. No.: 19/462,929

(22) Filed: Jan. 28, 2026

Related U.S. Application Data
(63) Continuation of application No. PCT/US2024/043867, filed on Aug. 26, 2024.
(60) Provisional application No. 63/581,226, filed on Sep. 7, 2023.

(51) Int. Cl. A61B 5/0215

(52) U.S. Cl. CPC A61B 5/0215; A61B 2562/125

(57) ABSTRACT

An implantable sensor device includes a deflectable diaphragm layer comprising vapor-deposited thin-film metal, a first capacitive electrode conformally formed on a first side of the deflectable diaphragm layer, and a second capacitive electrode coupled to a rigid substrate, the second capacitive electrode and the first capacitive electrode forming a variable capacitor. An implantable sensor device is manufactured by depositing a layer of thin-film metal on a substrate using a physical vapor deposition process and depositing a conformal layer of electrical conductor on a stack including the layer of thin-film metal.

### RELATED APPLICATIONS

This application is a continuation of International Patent Application No. PCT/US24/43867, filed Aug. 26, 2024, which claims the benefit of U.S. Provisional Patent Application Ser. No. 63/581,226, filed on Sep. 7, 2023, the complete disclosures of which are hereby incorporated by reference in their entireties.

### BACKGROUND

The present disclosure generally relates to the field of sensor devices. Some sensor devices, such as those suitable for medical implantation, can include deflectable diaphragms. Size, elasticity, compliance, biocompatibility, shape, and other features of such deflectable diaphragm components can impact suitability for implementation in implantable sensor devices.

### SUMMARY

Described herein are methods, systems, and devices that facilitate the transduction of pressure, such as blood/fluid pressure levels within a human body, into electrical signals for the purpose of sensing pressure. In particular, various pressure sensor packaging solutions are disclosed herein that provide for deposition of layer(s) of metal or other material(s) to form a diaphragm structure including one or more layer(s), at least one of which comprises/forms a conductive capacitive electrode (e.g., ‘anode’) layer. Such diaphragm structures/stacks can advantageously have a relatively thin profile and/or superelastic characteristics. For example, a sensor device in accordance with aspects of the present disclosure, which may serve as a biocompatible sensor implant device for cardiac or other implantation, may include one or more diaphragms formed of a thin, superelastic vapor-deposited layer, which may be formed of nitinol or similar material, wherein capacitive electrodes layer(s) is/are formed on the nitinol layer(s). The conductive electrode layer(s) can be disposed directly on the deposited thin-film, superelastic metal (e.g., nitinol) layer(s), or one or more dielectric/insulator layers can be formed between the electrode(s) and the superelastic metal layer(s). The thin-film superelastic metal and/or electrode layers can have certain topographical/surface features that advantageously increase the linear deflection and/or the effective surface area of the diaphragm on one or more sides thereof, such as corrugations, ridges, valleys, bumps, pillars, columns, spikes/pyramids, cones, clusters, and/or other geometric/uniform and/or amorphous/irregular features.

Examples of the present disclosure can include thin superelastic metal (e.g., nitinol) diaphragms having deposited/formed thereon one or more layers of high-k dielectric, wherein the superelastic metal layer provides a mechanical structure/substrate for dielectric and conductor stacking. The deposited thin-film metal layer can advantageously provide a superelastic, biocompatible face/shell for a sensor device.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silico, or overlaid on one or more of the physical components. Virtual elements can be presented on any combination of screens, headsets, holographically, projected, loudspeakers, headphones, pressure transducers, temperature transducers, or using any combination of suitable technologies.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates certain human anatomy showing example sensor implant locations in accordance with one or more examples.

FIG. 2 is a block diagram representing a system for wirelessly monitoring one or more physiological parameters associated with a patient in accordance with one or more examples.

FIGS. 3A and 3B show cross-sectional side views of a piezoresistive pressure sensor device in accordance with one or more examples.

FIGS. 4A and 4B show cross-sectional side views of a capacitive pressure sensor device in accordance with one or more examples.

FIGS. 5A and 5B show cross-sectional views of a sensor implant device including a pressure-transmitting medium in accordance with one or more examples.

FIG. 6 is a graph showing relationships between sensor diaphragm thickness, surface area, and sensitivity in accordance with one or more examples.

FIG. 7 is a block diagram showing a thin-film deposition system in accordance with one or more examples.

FIG. 8 shows a side cross-sectional schematic diagram of a sensor device having a capacitive electrode structurally conformal with a thin-film, superelastic diaphragm in accordance with one or more examples.

FIGS. 9-1 and 9-2 collectively provide a flow diagram illustrating a process for fabricating a capacitive electrode stack in accordance with one or more examples.

FIGS. 10-1, 10-2, 10-3, 10-4, and 10-5 show side cross-sectional schematic diagrams of a capacitive electrode stack/structure corresponding to various operations of the flow diagram of FIGS. 9-1 and 9-2 in accordance with one or more examples.

FIG. 11 shows various surface topologies that can be implemented for diaphragm and/or electrode layers of various sensor devices of the present disclosure in accordance with one or more examples.

FIG. 12 shows a schematic diagram of a wafer having a plurality of diaphragm structures formed thereon in accordance with one or more examples.

FIGS. 13A and 13B show front and back perspective views of an electrode-integrated sensor diaphragm structure in accordance with one or more examples.

FIG. 14 shows a base electrode structure for a sensor device in accordance with one or more examples.

FIG. 15 shows a sensor device including one or more diaphragm structures physically coupled to a base structure in accordance with one or more examples.

FIG. 16 shows electrical connectivity of components of a capacitive sensor device in accordance with one or more examples.

FIG. 17 shows a sensor having one or more corrugated diaphragms in accordance with one or more examples.

FIG. 18 shows a cross-sectional side view of a corrugated diaphragm having a conformal capacitive electrode in accordance with one or more examples.

FIG. 19 is a cutaway view of a human heart and associated vasculature showing certain catheter access paths for sensor device implantation procedures in accordance with one or more examples.

FIGS. 20A, 20B, and 20C show plan views of electrode-integrated sensor diaphragm structure designs in accordance with one or more examples.

FIG. 21A shows a plan view of a thin-film diaphragm sensor device in accordance with one or more examples.

FIGS. 21B and 21C show side exploded and bonded/assembled views, respectively, of a thin-film diaphragm sensor device in accordance with one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, various examples may be carried out in a manner that achieves or optimizes one advantage or group of advantages as taught herein without necessarily achieving other aspects or advantages as may also be taught or suggested herein.

Certain spatially relative terms, such as “outer,” “inner,” “upper,” “lower,” “below,” “above,” “vertical,” “horizontal,” “top,” “bottom,” “distal,” “proximal,” and similar terms, are used herein to describe a spatial relationship of one device/element or anatomical structure to another device/element or anatomical structure. It should be understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

The present disclosure relates to systems, devices, and methods for packaging devices configured for sensing and/or telemetric monitoring of one or more physiological parameters of a patient (e.g., blood pressure). Such pressure sensing/monitoring may be performed using cardiac implant devices having thin-film pressure sensor diaphragms with integrated and/or conformal capacitor electrodes. The term “thin-film” is used herein according to its broad and ordinary meaning, and may refer to any film, layer, sheet, skin, veneer, coating, covering, plating, enamel, finish, shell, overlay, or other type of membrane having a thickness ranging from a few nanometers (nm) to several micrometers (μm). Thin-film membranes of the present disclosure can be applied to a substrate during fabrication, and can be produced using various deposition techniques, including but not limited to sputtering, electrodeposition, thermal deposition, chemical vapor deposition (CVD), and physical vapor deposition (PVD). Formation of thin-film membranes in accordance with aspects of the present disclosure can involve producing a phase transition of a solid-state source material to gas and reconstituting the gas into a solid state during a deposition process. Thin-film membranes of the present disclosure can comprise any suitable or desirable material, including: metals such as gold, silver, platinum, titanium, nickel, copper, aluminum, and the like, and their alloys; alloys such as nitinol (nickel-titanium) or other superelastic material, NiTiCu (nickel-titanium-copper), AuSn (gold-tin), nickel-cobalt ferrous alloy (e.g., Kovar®, Ni29Co17Fe54), stainless steel, and the like; semiconductors such as silicon, gallium arsenide (GaAs), indium phosphide (InP), cadmium telluride (CdTe), and the like; oxides such as aluminum oxide (Al₂O₃), titanium dioxide (TiO₂), zinc oxide (ZnO), and the like; nitrides such as titanium nitride (TiN), silicon nitride (Si₃N₄), gallium nitride (GaN), and the like; polymers such as polytetrafluoroethylene (PTFE), polyimide, polyethylene terephthalate (PET), and the like; and ceramics such as silicon carbide (SiC), aluminum nitride (AlN), and the like. For example, thin-films can have thicknesses as small as 1 nm or as large as 10 μm, 15 μm, 20 μm, or larger, depending on the material properties desired. Membranes as disclosed herein may be formed using any type of sputtering process, including any process where atoms or molecules are ejected from a target material due to the bombardment by energetic particles, such as ions from a plasma, wherein the ejected particles travel through a vacuum or low-pressure gas environment and condense onto a target substrate, forming a thin membrane. “Sputtering,” as used herein, can cover any type of direct current, radio frequency, magnetron, or reactive sputtering, or any other vapor deposition process.

Sensor devices in accordance with aspects of the present disclosure can advantageously be packaged for long-term implantation in the cardiac environment, and therefore may have certain biocompatible features associated therewith. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Examples of capacitive pressure sensor devices disclosed herein can advantageously include electrode structures having one or more layers formed of physical vapor deposition and/or other thin-film deposition/formation processes. Such examples can advantageously combine multiple layers of superelastic deflectable substrates, dielectrics, and capacitive conductors/electrodes into a single, thin diaphragm structure suitable for implantation within the human body.

As described in detail below, implantable pressure sensors can be used to measure pressure levels in various conduits and chambers of body, such as in the various chambers of the heart. However, due to the accessibility and environmental conditions typically associated with the conduits/chambers of the heart and/or other potential sensor implant locations within a patient, only certain types of sensors and sensor packagings may be suitable for implantation for a given application. Examples of the present disclosure relate to the packaging of pressure sensor implant devices including certain electronics and telemetry features to allow for data and/or power communication wirelessly between the implanted sensor devices and one or more devices or systems external to the patient.

Aspects of the present disclosure relate to sensor devices, such as wireless implantable pressure sensor devices and other devices comprising deflectable diaphragm components. In particular, inventive features disclosed herein can be implemented in the context of implantable sensor devices, wherein integrated diaphragm features can advantageously provide biocompatible sealing and/or encapsulation of internal sensor components, such as capacitive electrodes and other circuitry, as well as other structural components of the device.

With respect to implantable pressure sensor devices, anatomical considerations can necessitate the use of sensor devices having relatively small form factors. For example, it may be desirable to implant sensor devices, such as pressure sensor devices, using transcatheter procedures, wherein the sensor device is advanced to the target implantation site through one or more venous or arterial blood vessels and/or various tortuous access paths. Examples of the present disclosure advantageously can be implemented in sensor devices having a sufficiently small profile/size to be transported by and/or within a catheter, sheath, or other instrument configured for transcatheter access/use. In addition to sizing constraints associated with implantable sensor devices (e.g., pressure sensor devices), sensor sensitivity and/or dynamic range requirements or desires likewise may drive sensor design. For example, with respect to pressure sensor devices, deflectable pressure diaphragms associated with such devices may be designed in a manner as to provide sufficient sensitivity to pressure conditions to which the device is exposed.

Furthermore, implantable sensor devices, such as diaphragm-equipped pressure sensor devices, may further need to provide biocompatibility and/or encapsulation characteristics suitable for in vivo implantation. For example, with respect to implantation within certain anatomy, such as within a chamber of a heart, or other fluid-filled anatomical vessel/chamber, such environments can present certain pressure, turbulence, and corrosion conditions, which may be associated with fluid/blood characteristics and/or cardiac cycling. Relative to non-implant environments, the human body represents a relatively harsh environment for electrical implant devices. Examples of the present disclosure provide sensor implant devices that provide extended-duration and/or lifetime hermetic seals/sealing, which can be advantageous and/or critical for implantable sensor application. For example, such hermetic sealing can prevent components of the environmental blood from degrading or otherwise interfering with the sensor and associated electronics. In addition, hermetic sealing of examples of the present disclosure can help prevent any non-biocompatible components of or associated with the sensor implant device from creating/causing toxic conditions within the body. Examples of the present disclosure furthermore provide sensor implant devices with ion-gas-deposited diaphragm layers/components that are relatively thin and provide hermitically-sealed, integrated sensor diaphragms. Such designs can have a reduced package thickness compared to certain other welded-metal solutions, which may include interfaces between metal and/or ceramic components thereof that occupy undesirable amounts of space. By integrating multiple functional components into a single thin outer layer/shell, the total package thickness/size can be minimized.

### Physiological Sensor Implant Locations

Certain examples are disclosed herein in the context of cardiac implant devices. However, although certain principles disclosed herein may be particularly applicable to the anatomy of the heart, it should be understood that sensor implant devices in accordance with the present disclosure may be implanted in, or configured for implantation in, any suitable or desirable anatomy. Furthermore, examples of the present disclosure may be utilized in non-biological environments as well.

The anatomy of the heart is described below to assist in the understanding of certain inventive concepts disclosed herein. In humans and other vertebrate animals, the heart generally comprises a muscular organ having four pumping chambers, wherein the blood flow therein is at least partially controlled by various heart valves, namely, the aortic, mitral (or bicuspid), tricuspid, and pulmonary valves. The valves may be configured to open and close in response to pressure gradients present during various stages of the cardiac cycle (e.g., relaxation and contraction) to control the flow of blood to respective regions of the heart and/or to blood vessels (e.g., pulmonary, aorta, etc.). The contraction of the various heart muscles may be prompted by signals generated by the electrical system of the heart, which is discussed in detail below.

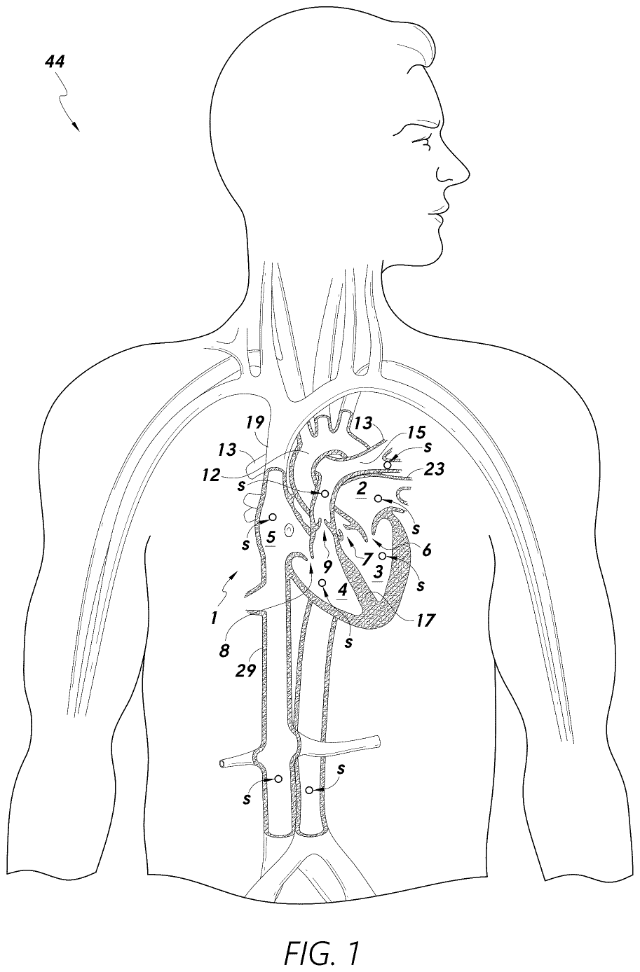

FIG. 1 illustrates an example representation of a heart 1 and associated anatomy having various features relevant to certain examples of the present inventive disclosure. The illustrated anatomy shows example implant locations for sensor devices in accordance with aspects of the present disclosure. Generally, the heart 1 includes four chambers, namely the left atrium 2, the left ventricle 3, the right ventricle 4, and the right atrium 5. In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11.

The pulmonary artery 11 carries deoxygenated blood from the right side of the heart to the lungs. Blood returns to the left atrium 2 from the lungs via the pulmonary veins 23. The pulmonary artery 11 includes a pulmonary trunk and left 15 and right 13 pulmonary arteries that branch off of the pulmonary trunk, as shown. In addition to the pulmonary valve 9, the heart 1 includes three additional valves for aiding the circulation of blood therein, including the tricuspid valve 8, the aortic valve 7, and the mitral valve 6. The tricuspid valve 8 separates the right atrium 5 from the right ventricle 4. The tricuspid valve 8 generally has three cusps/leaflets and may generally close during ventricular contraction (i.e., systole) and open during ventricular expansion (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (i.e., mitral and tricuspid) heart valves generally are coupled to a collection of chordae tendineae and papillary muscles (not shown) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

Health Conditions Associated with Cardiac Pressure and Other Parameters

As referenced above, certain physiological conditions or parameters associated with the cardiac anatomy can impact the health of a patient. For example, congestive heart failure is a condition associated with the relatively slow movement of blood through the heart and/or body, which causes the fluid pressure in one or more chambers of the heart to increase. As a result, the heart does not pump sufficient oxygen to meet the body's needs. The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Acute decompensated congestive heart failure is a leading cause of morbidity and mortality, and therefore treatment and/or prevention of congestive heart failure is a significant concern in medical care.

Various methods for identifying and/or treating congestive heart failure involve the observation of worsening congestive heart failure symptoms and/or changes in body weight. However, such signs may appear relatively late and/or be relatively unreliable. For example, daily bodyweight measurements may vary significantly (e.g., up to 9% or more) and may be unreliable in signaling heart-related complications. Furthermore, treatments guided by monitoring signs, symptoms, weight, and/or other biomarkers have not been shown to substantially improve clinical outcomes. Therefore, direct or indirect measurement/monitoring of pressure and/or other parameter(s) using implant devices can provide better outcomes than purely observation-based solutions. For example, without direct or indirect monitoring of cardiac pressure, it can be difficult to infer, determine, or predict the presence or occurrence of congestive heart failure or other pathologies. Treatments or approaches not involving direct or indirect pressure monitoring may involve measuring or observing other present physiological conditions of the patient, such as measuring body weight, thoracic impedance, right heart catheterization, or the like.

### Cardiac Pressure Monitoring

Cardiac pressure monitoring in accordance with examples of the present disclosure may provide a proactive intervention mechanism for preventing or treating congestive heart failure. Generally, increases in ventricular filling pressures associated with diastolic and/or systolic heart failure can occur prior to the occurrence of symptoms that lead to hospitalization. For example, cardiac pressure indicators may present weeks prior to hospitalization with respect to some patients. Therefore, pressure monitoring systems in accordance with examples of the present disclosure may advantageously be implemented to reduce instances of hospitalization by guiding the appropriate or desired titration and/or administration of medications before the onset of heart failure.

Dyspnea represents a cardiac pressure indicator characterized by shortness of breath or the feeling that one cannot breathe well enough. Dyspnea may result from elevated atrial pressure, which may cause fluid buildup in the lungs from pressure back-up. Pathological dyspnea can result from congestive heart failure. However, a significant amount of time may elapse between the time of initial pressure elevation and the onset of dyspnea, and therefore symptoms of dyspnea may not provide sufficiently-early signaling of elevated atrial pressure. By monitoring pressure directly according to examples of the present disclosure, normal ventricular filling pressures may advantageously be maintained, thereby preventing or reducing effects of heart failure, such as dyspnea.

Pressure sensor devices disclosed herein may be implanted in any of the chambers/vessels of the heart or other blood vessels (e.g., aorta, vena cava). FIG. 1 shows a number of example implantation sites for implantable sensor devices (denoted as ‘s’ in FIG. 1) in accordance with aspects of the present disclosure. For example, as shown in FIG. 1, sensor implant devices having integrated diaphragm components in accordance with the present disclosure can be implanted in the right atrium, right ventricle, left atrium, left ventricle, pulmonary arteries, inferior vena cava, aorta, or other anatomy. Generally, pressure elevation in the left atrium may be particularly correlated with heart failure, and so implantation of sensor implant devices in the left atrium may be desirable in some cases.

Left atrial pressure may generally correlate well with left ventricular end-diastolic pressure. However, although left atrial pressure and end-diastolic pulmonary artery pressure can have a significant correlation, such correlation may be weakened when the pulmonary vascular resistance becomes elevated. That is, pulmonary artery pressure generally fails to correlate adequately with left ventricular end-diastolic pressure in the presence of a variety of acute conditions, which may include certain patients with congestive heart failure. For example, pulmonary hypertension can affect the reliability of pulmonary artery pressure measurement for estimating left-sided filling pressure. Therefore, pulmonary artery pressure measurement alone may be an insufficient or inaccurate indicator of left ventricular end-diastolic pressure, particularly for patients with co-morbidities, such as lung disease and/or thromboembolism. Left atrial pressure may further be correlated at least partially with the presence and/or degree of mitral regurgitation.

In some solutions, pulmonary capillary wedge pressure can be measured as a surrogate of left atrial pressure. For example, a pressure sensor may be disposed or implanted in the pulmonary artery, and readings associated therewith may be used as a surrogate for left atrial pressure. However, with respect to catheter-based pressure measurement in the pulmonary artery or certain other chambers or regions of the heart, use of invasive catheters may be required to maintain such pressure sensors, which may be uncomfortable or difficult to implement. Furthermore, certain lung-related conditions may affect pressure readings in the pulmonary artery, such that the correlation between pulmonary artery pressure and left atrial pressure may be undesirably attenuated. As an alternative to pulmonary artery pressure measurement, pressure measurements in the right ventricle outflow tract may relate to left atrial pressure as well. However, the correlation between such pressure readings and left atrial pressure may not be sufficiently strong to be utilized in congestive heart failure diagnostics, prevention, and/or treatment. The present disclosure provides systems, devices, and methods for packaging implantable pressure sensors configured to provide direct measurements of pressure conditions at the implantation site.

Additional solutions may be implemented for deriving or inferring left atrial pressure. For example, the E/A ratio, which is a marker of the function of the left ventricle of the heart representing the ratio of peak velocity blood flow from gravity in early diastole (the E wave) to peak velocity flow in late diastole caused by atrial contraction (the A wave), can be used as a surrogate for measuring left atrial pressure. The E/A ratio may be determined using echocardiography or other imaging technology; generally, abnormalities in the E/A ratio may suggest that the left ventricle cannot fill with blood properly in the period between contractions, which may lead to symptoms of heart failure, as explained above. However, E/A ratio determination generally does not provide absolute pressure measurement values, but rather estimated information, which may not provide the requisite specificity in some cases. Furthermore, as ultrasound, or similar, imaging equipment is typically not found in the home environment, nor are typical patients competent to use such equipment, ambulatory devices in accordance with aspects of the present disclosure that are relatively easy to operate may be desirable in certain situations.

Cardiac pressure monitoring, such as left atrial pressure monitoring, can provide a mechanism to guide administration of medication to treat and/or prevent congestive heart failure. Such treatments may advantageously reduce hospital readmissions and morbidity, as well as provide other benefits. An implanted pressure sensor in accordance with examples of the present disclosure may be used to predict heart failure up to two weeks or more before the manifestation of symptoms or markers of heart failure (e.g., dyspnea). When heart failure predictors are recognized using cardiac pressure sensor examples in accordance with the present disclosure, certain prophylactic measures may be implemented, including medication intervention, such as modification to a patient's medication regimen, which may help prevent or reduce the effects of cardiac dysfunction. Direct pressure measurement in the left atrium can advantageously provide an accurate indicator of pressure buildup that may lead to heart failure or other complications. For example, trends of atrial pressure elevation may be analyzed or used to determine or predict the onset of cardiac dysfunction, wherein drug or other therapy may be augmented to cause reduction in pressure and prevent or reduce further complications.

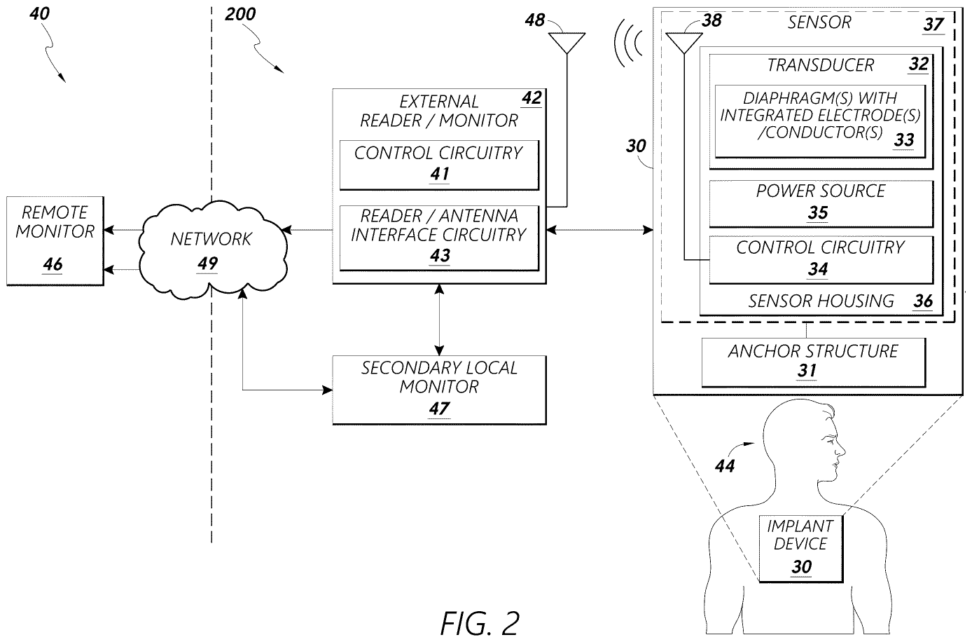

FIG. 2 is a block diagram representing a system 200 for wirelessly monitoring one or more physiological parameters associated with a patient according to one or more examples. FIG. 2 shows an implant device 30 comprising a sensor device 37, which may have associated therewith certain anchoring structure 31. For example, the anchoring structure 31 may be configured to anchor in and/or to one or more biological tissue walls. Although various examples of implantable sensor devices are illustrated and described in the present disclosure without separate anchoring structure, it should be understood that such omissions are solely for the purpose of clarity and any of the examples disclosed herein may have associated therewith certain anchoring structure for anchoring the device to biological tissue/anatomy at the implantation site.

The sensor device 37 may be a pressure sensor according to any of the examples disclosed herein. In some examples, the sensor 37 comprises a transducer 32, as well as certain control circuitry 34, which may be embodied in, for example, an application-specific integrated circuit (ASIC) and/or one or more passive devices (e.g., resistors, capacitors, inductors, etc.). The sensor device 37 further includes a diaphragm 33, which is formed of superelastic material and has layered/integrated thereon one or more electrode layers or other electronics that form part of the transducer circuit. The diaphragm 33 may be integrated at least in part with the outer layer(s) of the sensor housing 36. In some examples, the sensor housing 36 includes a radio-frequency-transparent structure that houses at least a portion of the antenna 38.

The control circuitry 34 of the sensor device 37 may be configured to process signals received from the transducer 32 and/or communicate signals associated therewith wirelessly through biological tissue using the antenna 38. The antenna 38 may comprise one or more coils or loops of conductive material, such as copper wire or the like, or piezoelectric resonator(s), or other wireless signal transmission component(s). In some examples, at least a portion of the transducer 32, control circuitry 34, and/or the antenna 38 are at least partially disposed or contained within the sensor housing/packaging 36 structure, which may comprise any type of material and may advantageously be at least partially hermetically sealed. The housing 36, as well as the diaphragm 33, may be formed at least in part using vapor deposition, as described in greater detail below.

The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further comprise one or more, storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in examples in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The housing/packaging 36 may comprise one or more tubes, cans, substrates/boards or other structures comprising glass, epoxy, ceramics, metal, and/or other rigid material(s) in some examples, which may provide mechanical stability and/or protection for the components housed therein. In some examples, the housing/packaging 36 is at least partially flexible. For example, the housing/packaging may comprise polymer or other flexible structure/material, which may advantageously allow for folding, bending, or collapsing of aspects of the sensor 37 to allow for passage thereof through a catheter or other introducing means. However, examples of the present disclosure can advantageously be implemented in such as manner as to provide long term hermetic protection, wherein thin-film, flexible diaphragm(s) for pressure transduction and associated electrical circuitry are integrated into a single part, component, and/or device.

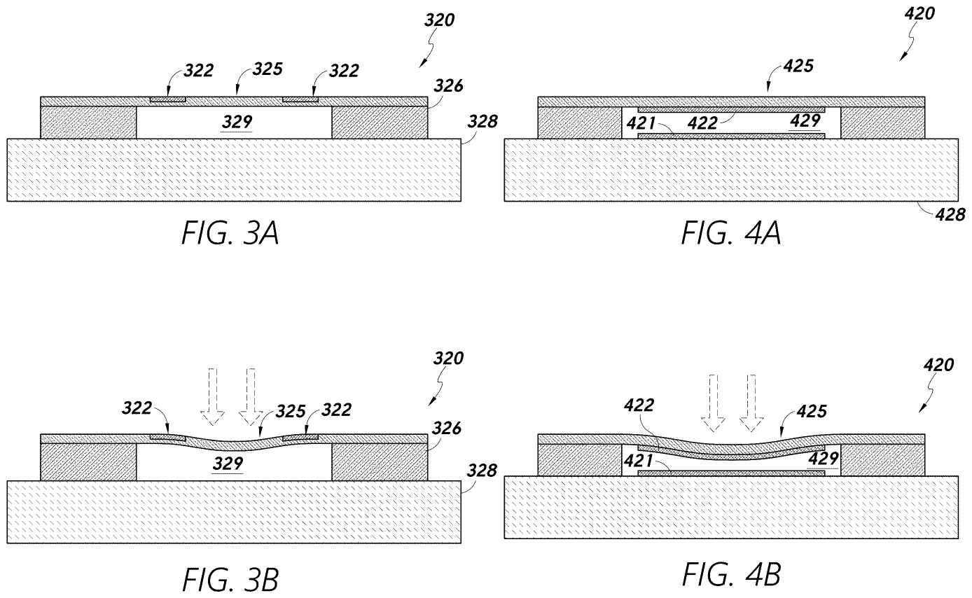

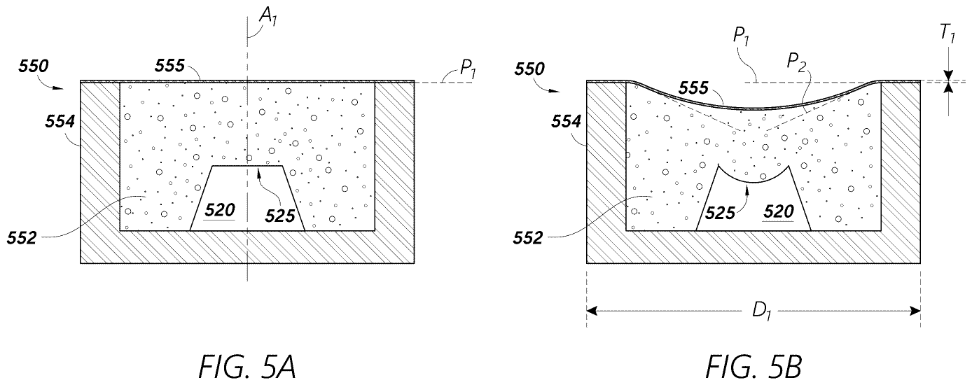

The transducer 32 may comprise any type of sensor means or mechanism. For example, the transducer 32 may be a force-collector-type pressure sensor. The transducer 32 is shown and described as comprising one or more diaphragms. However, it should be understood that pressure sensor devices disclosed herein may utilize any type of deflectable strain- or deflection-measuring component(s) configured to measure strain or deflection applied over an area/surface thereof, such as one or more pistons, bourdon tubes, bellows, or the like. The transducer 32 may be associated with the housing/packaging 36, such that at least a portion thereof is contained within or attached to the housing/packaging 36. In some examples, the electrode-integrated diaphragm 33 can serve as a component of a piezoresistive MEMS pressure sensor, which may be configured to use bonded or formed conductors to detect strain due to applied pressure, wherein resistance increases as pressure deforms the component/material. That is, the integrated electrode(s) 33 of the transducer 32 may be components of a piezoresistor. In such implementations, the conductor(s) may be applied to a thin-film nitinol diaphragm layer; piezoresistive pressure sensors are described below in connection with FIGS. 3A and 3B. Alternatively, the diaphragm 33 may be a component of a capacitive pressure sensor, where a capacitive plate/electrode layer is applied to the nitinol diaphragm layer, as described in detail throughout the present disclosure; capacitive pressure sensors are described generally below in connection with FIGS. 4A and 4B.

In some examples, the transducer 32 comprises or is a component of an electromagnetic pressure sensor, which may be configured to measure the displacement of a diaphragm by means of changes in capacitance, linear variable displacement transducer (LVDT) functionality, Hall Effect, or eddy current sensing. In some examples, the transducer 32 comprises or is a component of a piezoelectric strain sensor. For example, such a sensor may determine strain (e.g., pressure) on a sensing mechanism based on the piezoelectric effect in certain materials, such as quartz. In some examples, the transducer 32 comprises or is a component of a strain gauge. In any of such implementations, the relevant sensor electrodes/conductors may be applied to a thin-film nitinol diaphragm component, as described in detail herein.

The transducer 32 may be integrated with, or comprise, one or more layers of vapor-deposited, biocompatible material, as described in detail below. In some examples, the transducer(s) 32 is/are electrically and/or communicatively coupled to the control circuitry 34, which may comprise one or more application-specific integrated circuit (ASIC) microcontrollers or chips. The control circuitry 34 can further include one or more discrete electronic components, such as tuning capacitors, resistors, diodes, inductors, or the like.

In the system 200 of FIG. 2, the implant device 30 is implanted in a patient 44 for the purpose of monitoring one or more physiological parameters (e.g., left atrial pressure). The patient 44 can have the medical implant device 30 implanted in, for example, his/her heart (not shown), or associated physiology. For example, the implant device 30 can be implanted at least partially within the left atrium of the patient's heart.

In certain examples, the monitoring system 200 can comprise at least two subsystems, including the implantable internal subsystem or device 30 that includes the sensor transducer(s) 32, as well as control circuitry 34 comprising one or more microcontroller(s), discrete electronic component(s), and one or more power and/or data transmitter(s) 38 (e.g., antenna coils). The monitoring system 200 can further include an external (e.g., non-implantable) subsystem that includes an external reader 42 (e.g., coil), which may include a wireless transceiver that is electrically and/or communicatively coupled to certain control circuitry 41. In certain examples, both the internal and external subsystems include a corresponding coil antenna for wireless communication and/or power delivery through patient tissue disposed therebetween. The sensor implant device 30 can be any type of implant device. In some examples, the implant device 30 comprises a pressure sensor integrated with another functional implant structure, such as a prosthetic shunt or stent device/structure, valves, clips.

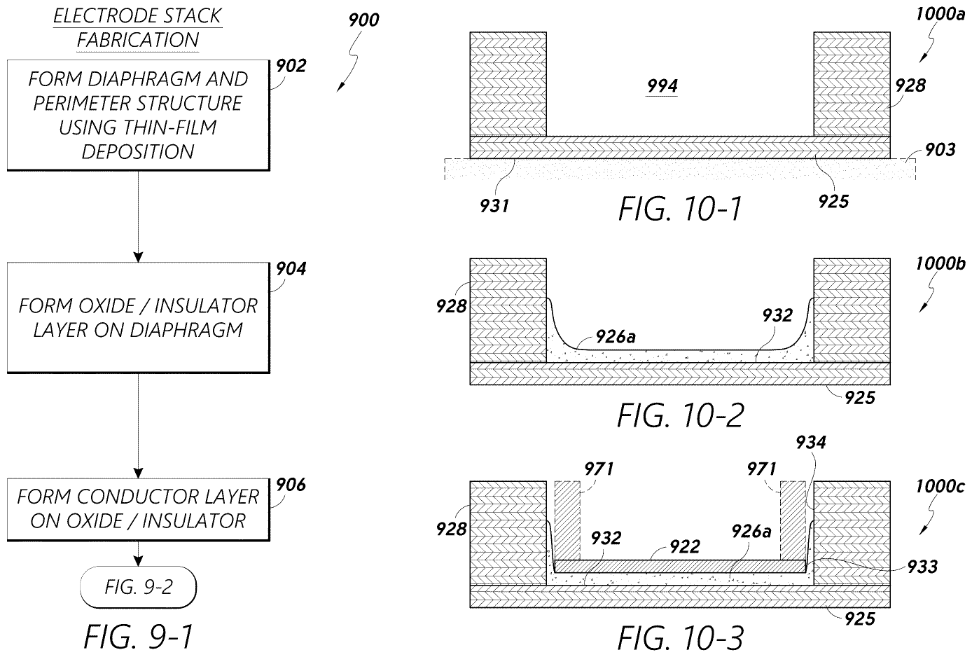

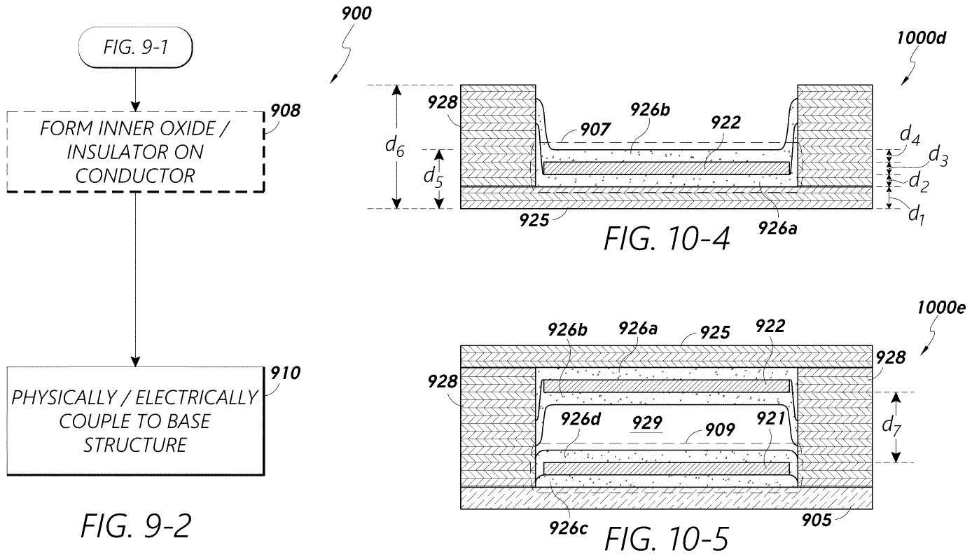

The implant device 30 can comprise certain anchoring structure 31, as referenced above. For example, the anchor structure 31 can include a percutaneously deliverable shunt device configured to be secured to and/or in a tissue wall. Although certain components are illustrated in FIG. 2 as part of the implant device 30, it should be understood that the sensor implant device 30 may only comprise a subset of the illustrated components/modules and can comprise additional components/modules not illustrated. The implant device 30 may represent an example of any of the implant devices shown in FIGS. 8-18, and vice versa.

In certain examples, the sensor transducer(s) 32 can be configured to generate electrical signals that can be wirelessly transmitted to a device outside the patient's body, such as the illustrated local external monitor system 42. The control circuitry 34 may comprise any type of transceiver circuitry configured to transmit an electromagnetic signal, wherein the signal can be radiated by the antenna 38, which may comprise one or more conductive wires, coils, plates, or the like. The control circuitry 34 of the implant device 30 can comprise, for example, one or more chips or dies configured to perform some amount of processing on signals generated and/or transmitted using the device 30. However, due to size, cost, and/or other constraints, the implant device 30 may not include independent processing capability in some examples.

The wireless signals generated by the implant device 30 can be received by the local external monitor device or subsystem 42, which can include a reader/antenna-interface circuitry module 43 configured to receive the wireless signal transmissions from the implant device 30, which is disposed at least partially within the patient 44. For example, the module 43 may include transceiver device(s)/circuitry.

The external local monitor 42 can receive the wireless signal transmissions and/or provide wireless power using an external antenna 48, such as a wand device. The reader/antenna-interface circuitry 43 can include radio-frequency (RF) (or other frequency band) front-end circuitry configured to receive and amplify the signals from the implant device 30, wherein such circuitry can include one or more filters (e.g., band-pass filters), amplifiers (e.g., low-noise amplifiers), analog-to-digital converters (ADC) and/or digital control interface circuitry, phase-locked loop (PLL) circuitry, signal mixers, or the like. The reader/antenna-interface circuitry 43 can further be configured to transmit signals over a network 49 to a remote monitor subsystem or device 46. The RF circuitry of the reader/antenna-interface circuitry 43 can further include one or more of digital-to-analog converter (DAC) circuitry, power amplifiers, low-pass filters, antenna switch modules, antennas or the like for treatment/processing of transmitted signals over the network 49 and/or for receiving signals from the implant device 30. In certain examples, the local monitor 42 includes control circuitry 41 for performing processing of the signals received from the implant device 30. The local monitor 42 can be configured to communicate with the network 49 according to a known network protocol, such as Ethernet, Wi-Fi, or the like. In certain examples, the local monitor 42 comprises a smartphone, laptop computer, or other mobile computing device, or any other type of computing device.

In certain examples, the implant device 30 includes some amount of volatile and/or non-volatile data storage. For example, such data storage can comprise solid-state memory utilizing an array of floating-gate transistors, or the like. The control circuitry 34 may utilize data storage for storing sensed data collected over a period of time, wherein the stored data can be transmitted periodically to the local monitor 42 or another external subsystem. In certain examples, the implant device 30 does not include any data storage. The control circuitry 34 may be configured to facilitate wireless transmission of data generated by the sensor transducer(s) 32, or other data associated therewith. The control circuitry 34 may further be configured to receive input from one or more external subsystems, such as from the local monitor 42, or from a remote monitor 46 over, for example, the network 49. For example, the implant device 30 may be configured to receive signals that at least partially control the operation of the implant device 30, such as by activating/deactivating one or more components or sensors, or otherwise affecting operation or performance of the implant device 30.

The one or more components of the implant device 30 can be powered by one or more power sources 35. Due to size, cost and/or electrical complexity concerns, it may be desirable for the power source 35 to be relatively minimalistic in nature. For example, high-power driving voltages and/or currents in the implant device 30 may adversely affect or interfere with operation of the heart or other body part associated with the implant device. In certain examples, the power source 35 is at least partially passive in nature, such that power can be received from an external source wirelessly by passive circuitry of the implant device 30, such as through the use of short-range, or near-field wireless power transmission, or other electromagnetic coupling mechanism. For example, the local monitor 42 may serve as an initiator that actively generates an RF field that can provide power to the implant device 30, thereby allowing the power circuitry of the implant device to take a relatively simple form factor. Such interrogation can be executed/performed intermittently/sporadically, quasi-continuously, and/or continuously. In certain examples, the power source 35 can be configured to harvest energy from environmental sources, such as fluid flow, motion, or the like. Additionally or alternatively, the power source 35 can comprise a battery, which can advantageously be configured to provide enough power as needed over the monitoring period (e.g., 3, 5, 10, 20, 30, 40, or 90 days, or other period of time).

In some examples, the local monitor device 42 can serve as an intermediate communication device between the implant device 30 and the remote monitor 46. The local monitor device 42 can be a dedicated external unit designed to communicate with the implant device 30. For example, the local monitor device 42 can be a wearable communication device, or other device that can be readily disposed in proximity to the patient 44 and implant device 30. The local monitor device 42 can be configured to continuously, periodically, or sporadically interrogate the implant device 30 in order to extract or request sensor-based information therefrom. In certain examples, the local monitor 42 comprises a user interface, wherein a user can utilize the interface to view sensor data, request sensor data, or otherwise interact with the local monitor system 42 and/or implant device 30.

The system 40 can include a secondary local monitor 47, which can be, for example, a desktop computer or other computing device configured to provide a monitoring station or interface for viewing and/or interacting with the monitored cardiac pressure data. The local monitor 47, monitoring performed thereby, and/or monitored data can be used/leveraged for troubleshooting. In an example, the local monitor 42 can be a wearable device or other device or system configured to be disposed in close physical proximity to the patient and/or implant device 30, wherein the local monitor 42 is primarily designed to receive/transmit signals to and/or from the implant device 30 and provide such signals to the secondary local monitor 47 for viewing, processing, and/or manipulation thereof. The external local monitor system 42 can be configured to receive and/or process certain metadata from or associated with the implant device 30, such as device ID or the like, which can also be provided over the data coupling from the implant device 30.

The remote monitor subsystem 46 can be any type of computing device or collection of computing devices configured to receive, process and/or present monitor data received over the network 49 from the local monitor device 42, secondary local monitor 47, and/or implant device 30. For example, the remote monitor subsystem 46 can advantageously be operated and/or controlled by a healthcare entity, such as a hospital, doctor, or other care entity associated with the patient 44. Although certain examples disclosed herein describe communication with the remote monitor subsystem 46 from the implant device indirectly through the local monitor device 42, in certain examples, the implant device 30 can comprise a transmitter capable of communicating over the network 49 with the remote monitor subsystem 46 without the necessity of relaying information through the local monitor device 42.

In certain examples, the antenna 48 of the external monitor system 42 comprises an external coil antenna that is matched and/or tuned to be inductively paired with the antenna 38 of the internal implant 30. In some examples, the implant device 30 is configured to receive wireless ultrasound power charging and/or data communication between from the external monitor system 42. As referenced above, the local external monitor 42 can comprise a wand or other hand-held reader. In some examples, the antenna 48 comprises a piezoelectric crystal.

Implantable Pressure Sensor Devices with Deflectable Diaphragms

Pressure sensors that can be used in medical implant applications include sensors utilizing micro-electromechanical system (MEMS) technology. Such devices may combine relatively small mechanical and electrical components on a substrate, such as silicon or other semiconductor substrate, and may incorporate deformable membranes that are used to measure pressure-induced deflection thereof, wherein the degree of deflection of the membrane is indicative of pressure conditions to which the sensor membrane is exposed at the implant location. Examples of the present disclosure improve upon certain MEMS technologies by applying conductor layers and/or other conductor features to deflectable diaphragm stacks formed on a diaphragm substrate comprising thin-film nitinol or similar material. Thin-film diaphragms of sensor devices disclosed herein may be constructed as components of any type of deflection-based sensor device, such as piezoresistive pressure sensors and capacitive pressure sensors. Such sensors advantageously include a flexible diaphragm layer that serves as a deformable membrane that deflects under pressure, wherein integrated conductor features of the diaphragm stack produce a mechanism to measure the displacement of the diaphragm(s). This structure can advantageously provide both transducer structure/functionality and protection from the hostile external environment.

With respect to resistive (e.g., piezoresistive) pressure sensors, certain conductive sensing elements may be fabricated directly onto the diaphragm of the device (or onto an insulator layer formed on the diaphragm) using sputtering, vapor deposition, or other application process, wherein changes in the electrical resistance of such conductor(s) can be determined to indicate a measure of pressure applied to the diaphragm. Generally, the change in resistance may be proportional to the strain on the conductor(s), wherein the change in resistance of the conductor(s) is related to the change in length of the conductor(s) induced by deflection of the diaphragm on which the conductor(s) are disposed.

FIG. 3A is a side view of a resistive pressure sensor device 320 implemented on a substrate 328 in accordance with one or more examples. FIG. 3B is a side view of the piezoresistive pressure sensor 320 of FIG. 3A, wherein a diaphragm 325 of the sensor is deflected in accordance with one or more examples. The deflection of the diaphragm 325 may be caused by pressure conditions to which the diaphragm 325 is exposed. The diaphragm 325 may be formed from a substrate material, such as thin-film nitinol, other material, which may be formed using physical vapor deposition or other process. In some examples, the thin diaphragm 325 may be formed by etching the substrate 326 to produce a relatively thin membrane for the diaphragm 325, which may enclose a cavity 329.

The diaphragm 325 may have one or more conductive traces or elements 322 disposed thereon and/or applied thereto. For example, the conductive elements 322 may comprise traces of metal or other electrical conductor, wherein one or more length portions of the conductor(s) extend over the diaphragm 325, such that deflection of the diaphragm 325 causes one or more portions of the conductor(s) 322 to elongate/lengthen, thereby altering the electrical resistance/impedance thereof. When the diaphragm 325 deflects, as shown in FIG. 3B, electrical current and/or voltage through the conductive element(s) 322 may be measured to determine respective resistances/impedances thereof, thereby providing a measurement indicating a degree of deflection of the diaphragm 325; such deflection indicates the environmental pressure experienced by the diaphragm 325.

FIG. 4A is a side view of a capacitive pressure sensor device 420 in accordance with one or more examples. FIG. 4B is a side view of the capacitive pressure sensor 420 of FIG. 4A, wherein a diaphragm 425 of the sensor is deflected in accordance with one or more examples. For capacitive pressure sensors having electrode-integrated diaphragm structures in accordance with the present disclosure, one or more conductive layers 422 may be deposited/applied on/to the thin-film nitinol diaphragm 425 to produce a capacitive electrode (e.g., anode). A corresponding electrode/plate 421 may be formed on an opposite-facing substrate 428, such that a cavity or other dielectric medium 429 is present between the electrodes/plates 422, 421. In some implementations, the electrode 421 provides a stationary/static electrode, while the diaphragm electrode 422 provides a flexible, dynamically-deflectable membrane electrode 422. With the area of such electrodes 422, 421 being fixed, the capacitance between the electrodes may be proportional to the distance(s) between them.

As shown in FIG. 4B, inward/downward deflection/deformation of the diaphragm 425 may change the spacing between the conductors 421, 422 over at least a portion of the diaphragm 425, thereby changing the capacitance of the capacitor formed between the diaphragm electrode 422 and the base electrode 422. Such change in capacitance may be measured by coupling the sensor device 420 to a tuned circuit, for example, which may have a fundamental frequency that is proportional to the degree of deflection of the diaphragm 425 and electrode 422.

Any of the various devices shown in FIGS. 3A/3B and 4A/4B can have certain oxide and/or other insulator layers (e.g., high-k dielectric) formed on electrode components to provide increased capacitance, reduced leakage current, improved breakdown voltage, and/or allow for reduced electrode/device size.

### Pressure Sensors Utilizing Pressure-Transmission Media

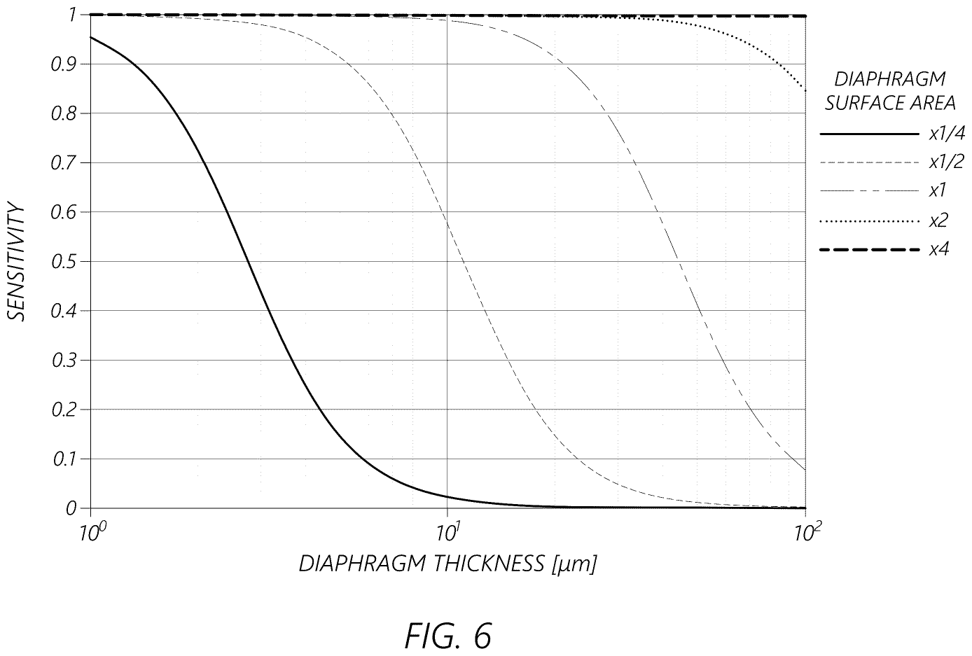

In some pressure sensor solutions, a sensor element, such as a MEMS pressure sensor, may be disposed within a housing enclosed in one area by a deflectable diaphragm, wherein a pressure-transmission fluid or other medium (e.g., oil, gel, epoxy) is disposed about the sensor element within the housing, such that external pressure causing inward deflection of the diaphragm is transferred to the pressure sensor for sensing thereof. FIGS. 5A and 5B show cross-sectional views of a sensor implant device 550 including a pressure-transmission medium 552 disposed within a housing 554 covered by a diaphragm 555 in accordance with one or more examples.

The diaphragm 555 may advantageously be deflectable, such that pressure conditions external to the enclosure 554 can cause inward deflection of the diaphragm 555 in a manner as to exert pressure on the sensor element surface/diaphragm 525. For example, the pressure-transmission medium 552 may comprise an incompressible fluid or medium in some examples. Alternatively, the medium 552 may be compressible, wherein deflection of the diaphragm 555 may cause a reduction in volume of the internal chamber of the can 554, thereby compressing the fluid/medium and resulting in increased pressure within the can 554 that is translated to the sensor element 520. The deflection of the diaphragm 555 may cause the diaphragm 555 to move from a non-deflected state or configuration in which the diaphragm lies in or primarily parallel to a transverse plane P₁ (e.g., transverse with respect to an axis of the diaphragm and/or sensor device) to a deflected state or configuration (see FIG. 5B) in which the diaphragm 555 conforms to a concave/deflected plane P₂ that is deflected relative to the transverse plane P₁ in a direction, e.g., toward the sensor element 520.

The pressure-transmission medium 552 is sealed within the housing 554 and disposed about the sensor element 520 within the outer enclosure. A portion of the enclosure 554 comprises the diaphragm component 555. While the use of pressure-transmission media for the purpose of transferring external pressure to an internally-housed pressure sensor device can be effective in terms of pressure reading, such implementations present certain downsides with respect to size and/or manufacturing complexity. For example, with respect to the sensor assembly 550, mechanical welding/filling may be necessary in areas where the transmission medium 552 is injected or applied in the housing 554. Risk of leakage of the transmission medium 552 can represent potential health hazards with respect to pressure sensors implanted within a human patient. Furthermore, the need for sensor components sufficiently large to house and seal the requisite volume of transmission medium 552 can impede the feasibility of low-profile designs that are desirable for sensor implants. As sensor device designs are reduced in size to allow greater flexibility with respect to minimally-invasive (e.g., transcatheter) delivery and implantation within the body, the materials and processes associated with such devices can require increased processing complexity, failure modes, and/or place ultimate limits on further reduction.

### Pressure Sensor Diaphragm Sizing/Sensitivity

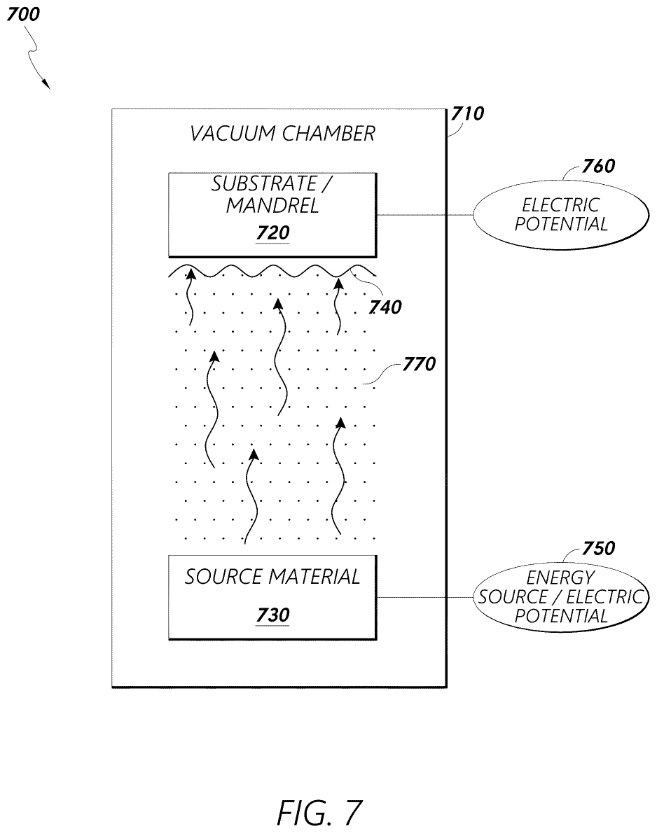

As referenced above, due to size constraints associated with implantable sensor devices, the area available for diaphragm components may likewise be constrained, depending on the design of the sensor device. As diaphragm effective areas reduce, it may be necessary to reduce the thickness T₁ of the diaphragm as well in order to maintain sufficient sensitivity in the diaphragm. For example, FIG. 6 is a graph showing relationships between sensor diaphragm thickness, surface area, and sensitivity in accordance with one or more examples. As demonstrated in the graph of FIG. 6, diaphragms having relatively smaller surface area generally must be relatively thinner in order to achieve comparable sensitivity compared to diaphragms having relatively greater surface area and otherwise similar design.

Processes implemented to form thin foils can cause variable strain hardening of the formed materials, which may result in relatively large variation in mechanical performance of a formed diaphragm. Furthermore, relatively thin materials utilized for diaphragm formation can require careful handling and assembly processes to weld the diaphragm structure to the larger sealed body. At such scale, the processes implemented can influence the material properties and mechanics of the diaphragm, thereby causing additional variation in diaphragm performance. In addition, raw wrought materials prior to forming can have defects and grain structures that are problematic at the micron scales of sensor diaphragms as disclosed herein.

In addition, certain sensor devices are designed with sensor diaphragms positioned/disposed at a distal end of the sensor device assembly. Since it can be advantageous to increase the area of the diaphragm to provide desirable sensitivity, as indicated in FIG. 6, increase in diaphragm area for such designs can be at the cost of increasing sensor device diameter/profile, potentially interfering with the ability to fit within a tubular catheter/shaft for delivery, particularly in consideration of diaphragm material thicknesses and deflection sensitivity according to the relationships demonstrated in the graph of FIG. 6. As sensor device designs evolve toward smaller and smaller-profile devices (e.g., millimeter-scale integrated implant devices), the ability to form and integrate such sensor device assemblies can become untenable with respect to axial-diaphragm designs. Thin-film nitinol diaphragm deposition as described in detail herein can facilitate transverse/lateral-facing diaphragms with respect to a long dimension/axis of a sensor device. Sensor designs including transverse/lateral diaphragms are illustrated and described in greater detail below in connection with examples of the present disclosure.

### Thin-Film Diaphragm Layer Deposition

Examples of the present disclosure advantageously provide solutions for utilizing vapor-deposited, thin-film nitinol (or similar) diaphragm components for implantable pressure sensors. Such thin-film diaphragms can advantageously facilitate the design of devices that are relatively small in size, while providing sufficient and/or improved sensor performance/sensitivity by allowing for diaphragm configurations that have sufficiently large area and/or material thickness characteristics to provide such sensitivity. Furthermore, thin-film deposited diaphragm structures disclosed in detail herein can facilitate integration of sensor electrodes with such diaphragm structures/stacks. Moreover, in some cases, suitable and/or improved biocompatibility characteristics and/or relatively simplified manufacturing processes can be provided through thin-film nitinol diaphragm deposition. Thin-film nitinol diaphragms can further be deposited in a manner such that the diaphragm(s) is/are integrated with other structural/mechanical housing/encapsulation component(s) of the associated sensor device in one or more uniform and integrated layers of deposited material. For example, while certain pressure sensor devices require manufacturing processes that involve multi-part and/or multiple-process manufacturing to seal/mechanically-couple diaphragm components to other structural components, examples of the present disclosure can allow for manufacturing without the need for such sealing/coupling step(s)/process(es) with respect to the diaphragm(s) and the adjacent structure of the device.

In some examples, sensor devices disclosed herein include plate structures having formed therein one or more deflectable diaphragms, as well as surrounding mechanical structure, wherein the plate is formed of thin-film vapor deposition. Such integration of the diaphragm(s) with at least some of the additional mechanical structure of a device can reduce the number of manufacturing steps/processes required for device fabrication, and furthermore can provide superior mechanical properties relative to certain non-integrated diaphragm solutions. Furthermore, integration of diaphragm and other mechanical structure of a device can reduce component count and process steps required to produce the resulting sensor packaging. With fewer components and areas requiring hermetic sealing, more robust protective housings can be produced that present a reduced risk of failure/leakage.

Certain examples of the present disclosure provide alternatives to wrought-metal machining, stamping, grinding, or the like, of sensor diaphragms in order to provide diaphragms with reduced thicknesses, improved sensitivity, and suitability for conformal electrode/conductor formation thereon. For example, processes for forming thin-file deposited diaphragm layers as described herein can provide the necessary precision and tolerances for micrometer-level layers, which can be difficult or impossible to produce using certain cutting and stamping processes. In some implementations, physical vapor deposition processing of sensor diaphragms as disclosed herein can produce nanometer-level precision and tolerancing. Such diaphragms may be advantageously formed using an ionized deposition process, rather than through stamping, welding, or other more complicated and/or inconsistent/error-prone processes. In some examples, diaphragms deposited/formed in accordance with aspects of the present disclosure comprise nitinol metal alloy rather than titanium, which may be utilized in other sensor designs.

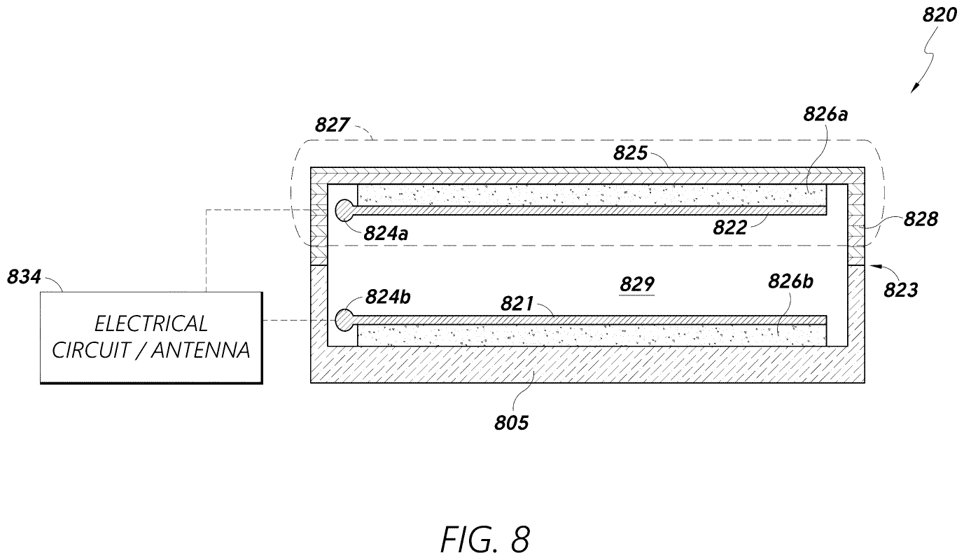

As referenced above, sensor diaphragms in accordance with aspects of the present disclosure may be manufactured/formed using ionized metal vapor deposition in some implementations. FIG. 7 is a block diagram showing a thin-film vapor deposition system 700 in accordance with one or more examples. Physical vapor deposition (PVD) and other vacuum deposition processes can be used to produce relatively thin films and coatings. In the system 700, a source material 730 (e.g., metal) transitions from a condensed phase to a vapor phase 770 and then back to a thin-film condensed phase 740 applied on/to a target substrate 720. Sputtering or evaporation may be implemented to produce the vaporized/plasma gas 770. The plasma gas 770 is deposited on the substrate 720 to form the layer 740 of the deposited source material. The vacuum chamber 710 may advantageously be devoid of air and particles that could otherwise interfere with the directed deposition onto the substrate 720.

Transformation from solid 730 to gas 770 can be achieved through the application of energy from an energy source 750. The energy source 750 may be any type of energy, including heat/thermal current, electrical current, and/or voltage potential relative to the potential 760 associated with the substrate 720. Energy may energize the source material 730 to produce the plasma form 770. The electric potential 760 relative to the source material 730 may serve to create a direction of the deposition flow 770 towards the substrate 720. Source material 730 may be positively charged in some cases, whereas the electric potential 760 of the substrate 720 may be negatively charged.

With respect to the various processes and devices disclosed herein, any type of deposition process, such as any of the deposition processes referenced or described herein, may be implemented to produce any of the inventive diaphragm components/layers and/or integrated conductor/electrode layers, including or as an alternative to physical vapor deposition. Examples may include cathodic arc deposition, in which a high-power electric arc is discharged at the target (source) material to blast away some into highly ionized vapor to be deposited onto the workpiece. For electron-beam physical vapor deposition implementations, the material to be deposited is heated to a relatively high vapor pressure by electron bombardment in a vacuum and is transported by diffusion to be deposited by condensation onto a relatively cooler workpiece. For evaporative deposition, the material to be deposited may be heated to a relatively high vapor pressure by electrical resistance heating in a vacuum. As another example, close-space sublimation can involve placing the source material and substrate in relatively close proximity to one another and radiatively heated. Pulsed laser deposition may be implemented by ablating the source material into a vapor using a high-power laser. Pulsed electron deposition may be implemented by ablating the source material to generate a plasma under nonequilibrium conditions using a highly energetic pulsed electron beam.

In some example examples, sputter deposition may be implemented, wherein a glow plasma discharge, which may be localized around the target substrate by a magnet, bombards the source material, thereby sputtering some away as a vapor for subsequent deposition. For sputtering applications, a magnetron may be employed that utilize strong electric and magnetic fields to confine charged plasma particles close to the surface of the sputter target. Generally, in a magnetic field, electrons follow helical paths around magnetic field lines, undergoing more ionizing collisions with gaseous neutrals near the target surface than would otherwise occur. The extra ions of the sputter gas created as a result of these collisions can lead to a higher deposition rate. The plasma can also be sustained at a lower pressure this way. The sputtered atoms are neutrally charged and so are unaffected by the magnetic trap. Other sputtering techniques that can be implemented include ion-beam sputtering, reactive sputtering, ion-assisted deposition, high-power impulse magnetron sputtering, gas flow sputtering, or the like.

### Thin-Film Diaphragm Layer Deposition

As described above, certain pressure sensor solutions can include sensors encapsulated in a rigid shell (e.g., metal shell/can) with a contained transmission fluid disposed between the external flexible diaphragm and an internal capacitive sensor device. Such solutions can be suitable for pressure sensors used for pressure transduction within a wet and/or corrosive environment, such as within the bloodstream. Examples of the present disclosure provide alternative solutions in which an external flexible diaphragm itself is used as the source of a capacitive (or resistive) electrical signal for pressure sensing due to integration with the diaphragm stack/structure of the conductive electrode(s). Although capacitor plate electrodes are disclosed in some contexts herein, it should be understood that any example disclosed herein is applicable also to resistive or piezoresistive conductor elements integrated with diaphragm stacks. For example, any of the capacitive sensor examples disclosed herein that are described as including flexible nitinol diaphragms with conformal capacitive plates/electrodes associated therewith can alternatively or additionally have resistive or piezoresistive sensor elements integrated/associated with the deflectable nitinol diaphragm. The manufacturing of such novel sensor solutions can be facilitated by materials and processing techniques that provide for thin-film deposition of nitinol, titanium, or the like, as a diaphragm substrate, wherein electrode conductor(s) can additionally be deposited in a similar manner on the diaphragm substrate (directly or on an insulator layer) to produce a diaphragm electrode stack, which can advantageously provide desirable profile for implant devices and suitable capacitive performance.

The ability to deposit superelastic nitinol in thin diaphragm layers, such as through physical vapor deposition as described above, can allow for the fabrication/construction of thin, flexible capacitive electrodes (e.g., anodes) directly/conformally on the diaphragm stack. Such implementations can produce sensor implant devices having relatively thin profiles, while providing suitable deflection and change in capacitance over an expected operational pressure range. For example, various electronic/conductive layers, components, and/or elements may be added to a diaphragm structure (e.g., stack) including a thin nitinol layer, thereby enabling the integration of passive and/or active electrical elements with relatively thin diaphragm structures. The application of electrical and/or insulative elements, such as conductive layers configured to serve as capacitive plates/electrodes in an electrical circuit, and associated dielectrics, to a nitinol or other thin-film diaphragm can be implemented through chemical or physical vapor deposition, sputtering, masking/etching, photolithography, screen/inkjet printing, electroplating, epitaxy, thermal oxidation, atomic layer deposition, anodization, or the like.

FIG. 8 shows a side cross-sectional schematic diagram of a sensor device 820 having a capacitive electrode 822 structurally conformal with a thin-film (e.g., less than 20 μm in thickness, such as between 5 μm-10 μm), superelastic diaphragm 825. As with any deflectable diaphragm layer(s) disclosed herein, the diaphragm layer(s) 825 may comprise any type of deposited thin-film, super-elastic metal material(s). Although deflectable diaphragm layers are described in certain contexts herein as comprising nitinol, it should be understood that such diaphragm layers can include any type of deposited (e.g., physical vapor deposition) thin-film (e.g., less than 15 μm), superelastic metal, including alloys such as nitinol (NiTi), NiTiCu, and the like. The utilization of physical vapor deposition of nitinol, titanium, gold, and/or other materials, as described in detail above, provides a basis for producing sensor diaphragm/electrode stacks/structures as disclosed herein that produce low-profile sensors suitable for implantation in the human body.

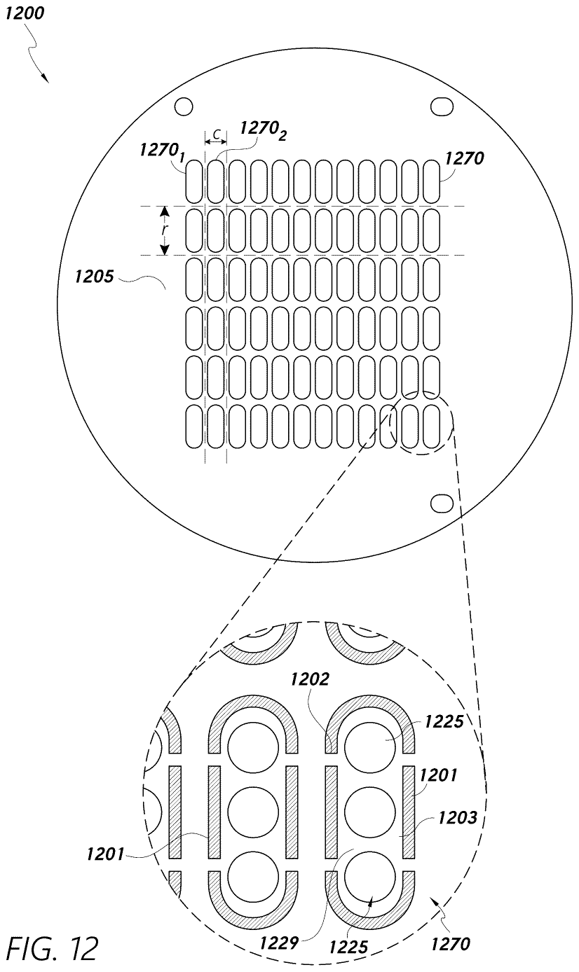

Transducing pressure inside the human body into capacitance can require relatively high, biocompatible, and stable diaphragm electrodes. To achieve the required compliance for a minimally-invasive implant device, constraining the thickness of a deflectable diaphragm stack/structure to a dimension in the order of microns can produce a suitable product. Furthermore, in order to achieve relatively large capacitance values measurable using either passive or active circuitry, relatively large diaphragm surface area may be necessary or desirable. However, such parameters can conflict with goals of achieving a minimally-invasive implant due to constraints of the lateral dimensions typically associated with such devices. As described in greater detail in connection with FIG. 11 below, a large surface area for nitinol and other metal layers associated with diaphragm electrode stacks of the present disclosure can be achieved using embedded three-dimensional surface features, such as trenches, spikes, fractals, and the like, implemented on one or more surface layers. The use of nitinol and similar materials for flexible diaphragm substrates on which capacitive electrodes can be disposed can provide a biocompatible layer that meets the above requirements and allows for production of low-profile, high-elasticity/capacitance sensor elements.

Unlike the device in FIGS. 5A and 5B, which includes a pressure-transmitting medium between the diaphragm and the sensor element, the device 820 of FIG. 8, as with other example(s) disclosed herein, may advantageously allow for pressure sensing without the need for a separate pressure-transmitting medium (e.g., incompressible fluid/oil), or a separate pressure sensor device/element apart from the diaphragm electrode(s) and companion electrode(s). That is, due to the integration of the capacitive electrode 822 and conformal disposition on the superelastic diaphragm 825, the flexible stack 827 (can be referred to as a thin-film ‘stack-up’) of the diaphragm 825 can serve as a capacitive electrode, without the need for additional transfer of pressure from the diaphragm 825 to a separate sensor element. Such implementations can be beneficial relative to oil-filled sensor devices, due to the space and complexity associated with such oil-filled devices. For example, the injection of oil into a pressure sensor chamber, and hermetic sealing/enclosure thereof, can be difficult to achieve and can present certain cost and safety complications. In the example of FIG. 8, as with other examples disclosed herein, the sensor 820 can be considered a dry capacitive sensor due to the absence of pressure-transmitting fluid, wherein the encapsulation of the sensor 820 (e.g., the outer nitinol shell/layer) itself forms a component of the pressure-transducing element, namely one plate/electrode of the capacitor element. That is, the outer nitinol layer/shell 825 provides both biocompatible/hermetic encapsulation, as well as pressure transduction into sensor electrical signals due to the integration therewith of the capacitive electrode 822.

The diaphragm electrode 822 combines with the corresponding electrode 821 to form a capacitor electrically coupled to the electrical circuitry 834 of the sensor 820. The capacitor plates 821, 822 can be electrically coupled to the resonance circuit of the circuitry 834 via certain electrical leads/connectors 824a, 824b, which may be integrated with the structure of the base substrate/structure 805 in any suitable or desirable manner, such as though various traces, vias, or the like. The nitinol diaphragm 825, which may have associated sidewalls/projections 828, can be physically sealed to a base structure/substrate 805 and a connection/joint 823 to provide a hermetically-sealed volume/space 829 between the capacitor plates 821, 822. The base substrate/structure 805 may comprise nitinol or other metal or material. The volume 829 may comprise a vacuum volume.

Certain implantable sensor solutions include thin layers of glass that serve as a substrate for a diaphragm. However, generally, the compliance of such glass layers can be inadequate with respect to producing an interpretable or desirably sensitive pressure sensor signal in a device comprising a relatively small surface area diaphragm. Therefore, the use of vapor-deposition nitinol diaphragm layers, which can advantageously provide greater compliance compared to glass and can generally be deposited in relatively thinner layers, in devices of the present disclosure can produce relatively low-profile sensor devices that still provide dynamic capacitive range to produce relatively high granularity in pressure sensor readings. In some implementations, diaphragm structures of examples of the present disclosure can include thin layers of nitinol, insulator, and metal/conductor capacitive electrode that collectively are thinner than a single layer of glass that has sufficient strength to meet biological implant requirements.

The nitinol layer 825 provides a thin, compliant, superelastic, biocompatible externally-facing shell for the device 820. Furthermore, the thin-film diaphragm 825 provides relatively large deflections and commensurately large variation in capacitance and signal amplitude, while remaining elastic. The conductor layer 822 may advantageously be insulated from the memory-metal structural diaphragm 825, to thereby electrically isolate the nitinol layer 825 from the electrical circuit of the capacitor 822/821. That is, the nitinol layer 825 may serve as a substrate for deposition/application of the conductor layer 822, wherein the conductor 822 is electrically isolated from the diaphragm 825 by the insulator layer 826a (e.g., oxide).

With further reference to solutions comprising deflectable capacitive sensor diaphragms formed of layers of glass, the use of nitinol as described herein in a thin-film application can provide additional benefits. For example, when comparing the elasticity of nitinol and glass in the context of a deflectable diaphragm, nitinol can be considered to provide superior superelasticity properties. Generally, nitinol can undergo substantial elastic deformation and revert to its original shape upon stress removal, which can be beneficial for applications demanding considerable degree and number of deflections. Conversely, glass, being a relatively brittle material, generally exhibits relatively low elasticity, such that it does not tolerate extensive strains efficiently and can fracture under high stress. Furthermore, the strength of glass substrates can depend on the surface finish of the glass, wherein flaws or cracks on the surface can act as stress concentrators, which can compromise its structural strength. Conversely, the nitinol diaphragm 825 can advantageously endure high stress and strain without succumbing to permanent deformation, whereas glass, due to its brittle nature, can be prone to catastrophic failure when subject to stress.

The thin-film nitinol diaphragm 825 provides additional benefits compared to glass diaphragms, including the ability to form shaped surfaces, such as corrugations or extrusions, in the diaphragm layer, which can increase the effective surface area of the diaphragm 825. Furthermore, formation of corrugations, extrusions, and/or other surface-topological features in diaphragm layer(s) of examples of the present disclosure (e.g., the diaphragm layer 825) can increase the linear deflection regime of the diaphragm. That is, such surface features can provide advantageous mechanical features/behavior for a thin-film diaphragm as disclosed herein. Corrugations and other surface formations can also allow for tuning of sensitivity and linearity of the capacitive sensor device. In addition such corrugations and other surface formations can provide thermal and package stress reduction and/or add shape to create a more stable shape during handing and assembly. It should be understood that any sensor diaphragm features disclosed herein, the formation of which can be enabled by the use of physical vapor deposition and similar process(es), can advantageously provide increased surface area and/or increased linear deflection. Glass, on the other hand, presents certain challenges when it comes to forming shaped surfaces therein due to the structural brittleness/fragility thereof and the lack of available processes to produce surface features precisely in glass surfaces. Nitinol can be tuned mechanically in ways that glass and other diaphragms cannot, and provides greater deflection to produce greater change in capacitance when implemented with conformal electrode layers described herein that cover substantial areas of the nitinol diaphragm. For corrugations in diaphragm layer(s) (e.g., diaphragm 825; diaphragm corrugation features described in detail below relative to various examples), such three-dimensional features may be formed by depositing thin-film metal on a surface/mold/mandrel having such surface features. With respect to fractal-like and/or porous surface features, formation of the same may involve masking, electroplating, or the like.

One or more of the dielectric layers 826a, 826b may comprise high-k dielectric material. The presence of the dielectrics 826, in addition to electrically isolating the plates 822, 821 from the physically proximate substrates 825, 805 to avoid corruption of sensor signals, can serve to protect the electrical circuit associated with the electrodes 821 822 from circuit break-down from capacitance between the plates 822 and the substrate(s) 825, 805, which may be at least partially conductive, as in the case of nitinol. Use of high-dielectric materials can impede the creation of a capacitance between the nitinol layer 825 and the electrode layer 822, reducing unwanted stray capacitance that might otherwise negatively impact the circuit. The nitinol layer 825, when the sensor device 820 is implanted, is exposed and facing the biological environment, whereas the electrodes 821, 822, are internal to the device 820.

The capacitance of the sensor 820 may be based at least in part on the area of the plates 822, 821. Therefore, by covering a substantial portion of the area of the nitinol diaphragm 825 with a conformal layer of the conductor 822, the capacitance of the device 820 can be maximized. For example, compared to certain solutions in which only a minority portion of a deflectable diaphragm corresponds to an area of a sensor capacitor plate of the device, the implementation of the device 820 in FIG. 8, wherein the dynamic/deflectable capacitor plate 822 covers a substantial area (e.g., more than half of the area) of the diaphragm 825, can produce greater capacitance range per diaphragm area.

The capacitor electrodes 821, 822 are electrically coupled to certain electrical circuitry 834, including an antenna configured to facilitate wireless transmission of sensor signals and/or signals derived therefrom. In some implementations, the circuitry 34 comprises active circuit components, including amplifiers or the like configured to convert capacitance of the plates 821, 822 into readable signals. Collectively, the capacitor formed by the plates 821, 822 can be electrically coupled to the antenna in a manner such that changes in the capacitance of the capacitor produces resonance changes in the antenna, wherein such resonance of the antenna can be decoded to determine pressure levels causing the resulting capacitance.

FIGS. 9-1 and 9-2 collectively provide a flow diagram illustrating a process 900 for fabricating a capacitive electrode stack in accordance with one or more examples. FIGS. 10-1, 10-2, 10-3, 10-4, and 10-5 show side cross-sectional schematic diagrams of a capacitive electrode stack/structure 1000 corresponding to various operations of the flow diagram of FIGS. 9-1 and 9-2 in accordance with one or more examples.

At block 902, the process 900 involves forming a diaphragm 925 and perimeter structure 928 using thin-film deposition (e.g., PVD) or other technology. For example, such diaphragm and perimeter layer(s) may comprise nitinol or other shape-memory alloy. The layering associated with blocks 902 may advantageously produce a superelastic diaphragm structure 1000a.

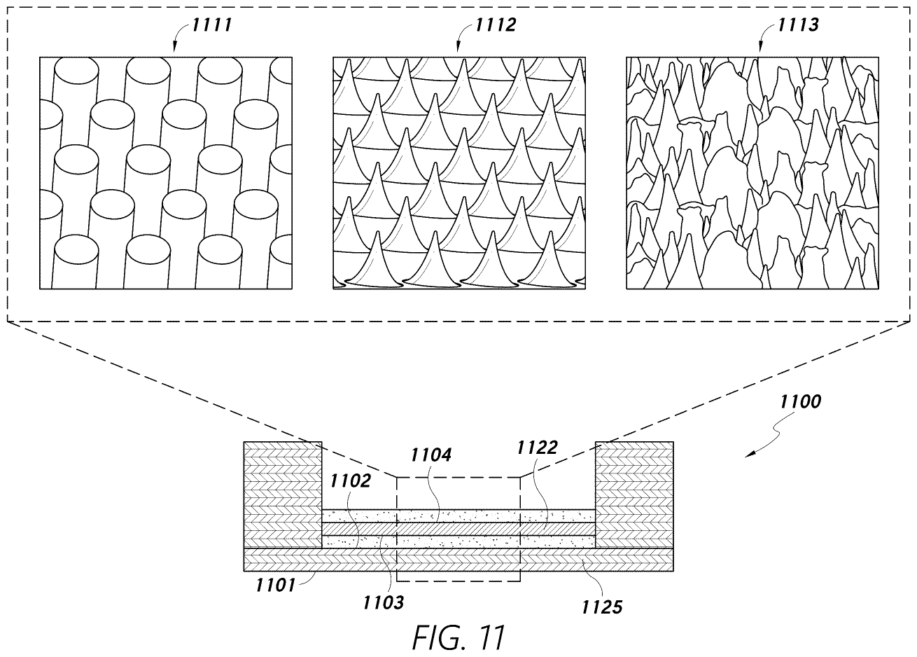

FIG. 10-1 shows the structure 1000a including the formed diaphragm 925, as well as supporting side structures 928, which may provide spacing for the diaphragm 925. One or both of the diaphragm 925 or support structures 928 may be formed of nitinol or other superelastic material through vapor deposition, as described in detail herein. The diaphragm 925 may be formed by depositing the layer(s) thereof on/against a mandrel or other substrate or shaping form 903. Such shaping form 903 may have a generally flat surface, and/or may include certain surface features configured to produce three-dimensional surfacing on the outer surface 931 of the diaphragm 925.

The volume/space 994 within the diaphragm 925 and support structures 928 may be formed through trenching or other mechanism in some implementations. For example, in such implementations, at the bottom of the trench, an electrode stack may be layered on top of the diaphragm thin-film 925. In some implementations, the support structure(s) 928 may be added to a base structure 905 (see FIG. 10-5) and project therefrom in addition to, or as an alternative to, projecting from the diaphragm layer 925.

At block 904, the process 900 involves forming an oxide or other insulator layer 926a on the inside surface 932 of the diaphragm 925. Although described in some contexts as oxide layer(s), it should be understood that any insulator/dielectric layers described herein may comprise non-oxide dielectric materials that allow flexing. For example, such layers may comprise polyimide, perylene, or the like. In some implementations, various oxide layers can be sandwiched between non-oxide layers, such as polyimide, perylene, or the like. FIG. 10-2 shows the diaphragm structure 1000b including the one or more layers 926a of oxide disposed on the inner surface 932 of the diaphragm 925. As illustrated, the oxide/insulator 926a may be contained at least partially within the raised side structures 928. The diaphragm 925 and the side structure(s) 928 may form a can structure in which additional layering may be deposited/formed. In some implementations, the oxide/insulator layer(s) 926 may be deposited against the inner sidewalls of the raised side structures 928. As with any oxide/insulator/dielectric layer disclosed herein, the layer 926a may comprise a single oxide layer, multiple oxide layers, a layer of perylene, alternating layers of flexible coatings like perylene and oxides, or other similar layering and/or composition.

In some implementations, a native oxide layer may form on the inside surface 932 of the nitinol diaphragm 925, wherein such oxide layer can provide some amount of insulation between the nitinol 925 and a subsequently formed/deposited layer (e.g., conductor/electrode layer). However, the native oxide layer, which generally may have a relatively low k-value, may not sufficiently insulate the subsequently applied conductor 922 (see FIG. 10-3) from the diaphragm 925. Rather, the application of a high-k dielectric, such as through sputtering or other process, can provide the desired insulation for the electrical circuit without requiring undesirable thickness/profile.

The oxide layer 926a may be sputtered onto the inner surface 932 of the diaphragm 925. In some implementations, the process 900 involves creating gas molecules of hafnium oxide, zirconium oxide, aluminum oxide, titanium oxide, or other high-k dielectric, which may be directed using electric fields onto the surface 932 of the substrate 925. The oxide 926 may be deposited layer-by-layer to achieve the desired thickness D2 of the oxide layer 926a. In some implementations, the oxide 926a can be deposited using chemical vapor deposition, wherein a precursor gas that contains a desired oxide is introduced into a chamber with the substrate 925 disposed therein. The gas may react on the surface 932 to form the oxide layer 926a. Such processes may be suitable for materials that do not oxidize naturally to a sufficient degree, or when a specific oxide/insulator thickness or quality is desired. In some implementations, physical vapor deposition or atomic layer deposition may be implemented to deposit one or more separate layers of oxide 926 onto the substrate 925. In some implementations, insulator/oxide layer(s) 926 may run up the side walls 928 to insulate the electrode 922 from the side wall(s) 928, which can serve to reduce stray capacitances.

At block 906, the process 900 involves forming a layer of electrical conductor 922 over the oxide 926a. FIG. 10-3 shows the diaphragm/electrode stack 1000c with the capacitive electrode 922 applied thereto. The structure 1000c may comprise one electrode of a capacitive sensor, such as an anode (or cathode) thereof.

In some implementations, the process 900 further involves forming electrical contacts 971 to the electrode 922. For example, the contact(s) 971 may contact and stand-off of the electrode layer 922 to provide electrical communication with the capacitive plate 922. The contact(s) 971 may be dimensioned to provide a projection structure that comes into contact with a corresponding contact/lead of a base structure when the diaphragm structure 1000c is coupled to a base (see FIG. 10-5) to form an enclosed sensor device. Although shown in FIG. 10-3 as deposited or otherwise formed (remaining structure from an etching process) directly on the inside surface of the electrode 922, it should be understood that the electrical contact(s) 971 to the electrode 922 may be configured in any manner or in/on any structure of the stack 1000c.

Although the processes 900 is described and illustrated as involving the formation of the conductor layer 922 over the oxide layer(s) 926a, in some implementations, the conductor 922, which serves as the capacitive electrode (e.g., anode plate) for a capacitive pressure sensor, may be formed directly on the inner surface 932 of the diaphragm 925. However, the implementation of the oxide layer 926a may allow for electrical isolation between the conductor 922 and the diaphragm 925, which may be desirable to facilitate proper functionality of the associated electrical circuit. In some implementations, a gap may be formed between the lateral surface 933 of the conductor 922 and the inner boundary 934 of the support structure(s) 928, which may facilitate electrical isolation between the conductor 922 and the support structure(s) 928. For example, where the support structures 928 are in contact with and/or integrated with the diaphragm 925, contact between the capacitive electrode 922 and the side structures 928 can electrically short/shunt the conductor 922 to the body of the sensor to some degree. The conductivity of the nitinol diaphragm 925 and side support structures 928 may be significantly lower than that of the electrode conductor 922, which may comprise gold (Au), platinum (Pt), or other conductor metal. In order to produce a sufficiently compliant diaphragm 925, it may be desirable for the nitinol layer 925 to be relatively thin. Therefore, physical vapor deposition or other technology that allows for the deposition of plates/layers in the order of microns may desirably be implemented in connection with the process 900. Such fabrication can produce relatively high elastic movement, which transduces to a relatively large range in capacitive signal in operation.

The thin layer 925 of nitinol can advantageously be used for pressure transduction through the pressure-sensitive mechanism thereof, while still allowing for the electrode 922 placed thereon, in combination with the nitinol diaphragm 925, to be relatively thin so as to not interfere with the mechanics of the deflectable diaphragm 925. In some implementations, both the nitinol layer 925 and the electrode layer 922 are deposited using physical vapor deposition, rather deposition or sputtering processes.

In some implementations, the process 900 involves forming a second layer 926b of insulator/oxide over the conductive electrode 922, as shown in block 908. The stack 1000d shown in FIG. 10-4 shows the inner/secondary insulator layer(s) 926b covering at least a portion of the electrode 922 on an inward-facing side thereof. In some implementations, the second dielectric layer 926b is omitted. In the illustrated example of FIG. 10-4, the dielectric layers 926a, 926b isolate the conductor 922 from the nitinol layer 925, which serves as the compliant mechanical mechanism for the electrode structure 1000d.

The layers of the electrode stack 1000d may have any suitable or desirable thicknesses. For example, in some implementations, the exterior nitinol diaphragm layer 925 may have a thickness d₁ about 5 μm. In some implementations, the entire stack thickness d₅ may be less than 10 μm, such as about 6 μm or less. Additional example thicknesses for the various layers of the bioelectronic structure/platform 1000d shown in FIG. 10-4, and similar structures disclosed herein including insulated and metallized diaphragms, include: for the bottom oxide layer 926a (e.g., hafnium oxide), such layer(s) may have a thickness d₂ of less than 500 nm, such as about 300 nm (e.g., less than 350 nm), or less (e.g., about 250 nm, 200 nm, 150 nm, 100 nm, or less); for the capacitive conductor layer 922, such layer(s) may have a thickness d₃ of less than 500 nm, such as about 350 nm (e.g., less than 400 nm), or less (e.g., about 300 nm, 250 nm, 200 nm, 150 nm, 100 nm, or less); for the second insulation layer 926b, such layer(s) may have a thickness d₄ of less than 200 nm, such as about 100 nm (e.g., less than 150 nm), or less (e.g., about 90 nm, 80 nm, 70 nm, 60 nm, or less).

At block 910, the process 900 involves physically and electrically coupling the electrode stack 1000d to a base structure 905. The structure 905 may have any suitable or desirable composition, and may or may not be electrically conductive.

The base 905 may have a stack structure 909 formed thereon that is similar in one or more aspects to the stack structure 907 of the diaphragm structure 1000d. The base stack 909 can include a second capacitive electrode 921 (e.g., cathode), wherein the capacitance between the electrode 922 and the electrode 921 changes as the diaphragm 925 deflects, such that, in operation, the conformal electrode 922 dynamically deflects, while the electrode 921 is a physically static electrode. FIG. 10-5 shows the combined sensor capacitor structure 1000e. In some implementations, both electrodes 921, 922 are formed on flexible diaphragms, such that both the electrode and counter-electrode are dynamically movable. For example, with respect to the device 1000e of FIG. 10-5, in some implementations, both the layer 925 and the layer 905 are flexible and/or formed of thin-film, vapor-deposited material.

The bonding of deflectable diaphragm stack 1000d with the base 905 and associated electrode stack can produce the combined capacitor, as well as a hermetic seal. The stack 1000e can create a pressure sensitive sensor capable of sustaining relatively large deflections while allowing for large changes in capacitance between the plates 922, 921. The change in capacitance of the plates 921, 922 in response to deflection of the diaphragm 925 can be read-out using passive and/or active circuitry electrically and/or communicatively coupled to the electrode elements 921, 922. In some implementations, certain electrical connectivity features associated with the base 905 are coupled the electrodes 922, 921 to provide a closed electrical circuit. In some implementations, either the top oxide layer 926b or the top oxide layer 926d is included, but not both. That is, the device 1000e may function as desired if either the cathode or the anode contain a top oxide layer that is disposed in the area between the electrodes 921, 922, at least in part.

In some implementations, a vacuum or air volume/space 929 is present between the electrode stacks 907, 909. With further reference to the capacitive electrode 922 formed on the diaphragm 925, when the nitinol diaphragm 925 deflects, such deflection may in-turn cause commensurate deflection in the conformal electrode 922, thereby approximating the electrode 922 in one or more areas thereof (e.g., particularly in a center area of the electrode 922) to the paired corresponding capacitive plate/electrode 921.

The diaphragm structure 1000d may be bonded/coupled to the base structure 905 in any suitable or desirable manner, such as through welding, flip-chip bonding, adhesion, or other coupling means. The base electrode stack 909 may include one or more oxide layers 926c, 926d, or alternatively, one or more of such oxide layer(s) may be omitted. The combined structure 1000e may advantageously be hermetically sealed, such that the electrodes 922, 921, and associated electrical connectors/circuitry may be protected from the external environment of the device 1000e.

The electrode 921 can be built onto the body structure/substrate 905, whereas the electrode plate 922 is associated with the deflecting diaphragm/plate structure 925. The base structure 905 can comprise printed circuit board, silicone, plastic, ceramic, glass, or other at least partially rigid material. The height d₆ of the diaphragm structure 1000d may be controlled to produce the desired plate spacing d₇. Such spacing may be controlled at least in part by controlling the thickness of the perimeter structure 928 to produce the desired base capacitance.

The combined device 1000e can be used to transduce pressure outside of the device into capacitance of the plates 922, 921. Generally, as described in detail above, transducing pressure into capacitance requires a relatively highly compliant and stable diaphragm with high tolerance and precise dimensions. Using physical vapor deposition of nitinol for the diaphragm 925 can offer a biocompatible layer that meets such requirements. Implementing the thin superelastic diaphragm 925 with the integrated insulation 926 and electrode 922 layers stacked opposite-facing with respect to the stable electrode 921, which may have similar or different stacking/structure, can produce a device where an external pressure on the deflectable diaphragm 925 displaces the diaphragm 925 and electrode 922, causing a measurable change in capacitance thereof.

In addition to implementing the conformally-coated diaphragm having high-k dielectric layer(s) 926 stacked with the capacitive electrode 922 to produce a thin-film stack, the nitinol layer 925 and/or electrode layer 922 may contain certain three-dimensional surface features on one or more areas thereof that provide an increase in the effective surface area of such components.

FIG. 11 shows various surface topologies that can be implemented for diaphragm and/or electrode layers of sensor devices of the present disclosure in accordance with one or more examples. FIG. 11 shows a diaphragm/electrode stack/structure 1100 in accordance with examples disclosed herein, including a nitinol diaphragm 1125 having a conformal capacitive electrode 1122 formed thereon. In some implementations, a surface of the electrode 1104 (e.g., inner surface 1104) or nitinol diaphragm 1125 (e.g., outer 1101 or inner 1102 surface) may have certain surface protrusion/projection features associated therewith. For example, the surface(s) may be roughened, scuffed, etched, or otherwise texturized to increase the surface area of the electrode 1122 (e.g., by one or two orders of magnitude, or more), without necessarily increasing the lateral/planar surface area covered by the electrode. Such texturization can allow for the use of relatively small capacitive diaphragms with relatively high surface area due to the topology/texture of the electrode surface.

The protrusion/projection features of the layer surface(s) can be formed using various processes. In some implementations, extrusions may be formed in the Nitinol layer 1125 to create a surface having increased surface area. For example, any number or pattern of trenches, pillars, needles, columns, or the like may be formed in the nitinol diaphragm surface. Corresponding, e.g., concentric rings/shapes that have a similar structure may be formed/present on the corresponding electrode of the sensor. Example column/pillar features 1111 are shown in FIG. 11. Such extrusions additionally or alternatively may be implemented on one or more sides of the conductor layer 1122 as well. Increased surface topology of the layer(s) of the stack 1100 can be created through trenching/etching to produce lateral surfaces in the extrusions in the areas etched away from the base material.

In some implementations, electroplating may be implemented to create a fractal-like surface 1113 providing increased surface area. For example, such electroplating may be implemented to form protrusions 1113 on the surface of the electrode. Electrochemical processes can produce plating extrusions. Alternatively, etching or growth processes can be implemented to form surface-area-enhancing extrusions on one or more surfaces of the electrode stack. In some implementations, cone/pyramid-type forms 1112 may be formed through masking using certain deposition processes. The formation of surface-area-enhancing extrusions can be through additive or subtractive processes. Any of the surface texturing/topology patterns, formations, and/or configurations shown and/or described may be implemented on either side (1101, 1102, 1103, 1104) of either of the electrode 1122 or nitinol diaphragm 1125.

With reference back to the process 900 of FIGS. 9-1 and 9-2, surface-topological features may be formed on/in either the thin-film diaphragm 925 and/or the electrode conductor 922 at any point in the process 900. For example, surface features/formations may be implemented prior to the formation/addition of the oxide/insulator layer 926b in connection with block 908 (layer 926b shown in FIG. 10-4).

Any of the features 1111, 1112, 1113, as well as any conductor, memory-metal, insulator, oxide, dielectric, or other, e.g., thin-film, layers described herein can be formed of any type of additive deposition/deposited process, which may be implemented with or without masking.

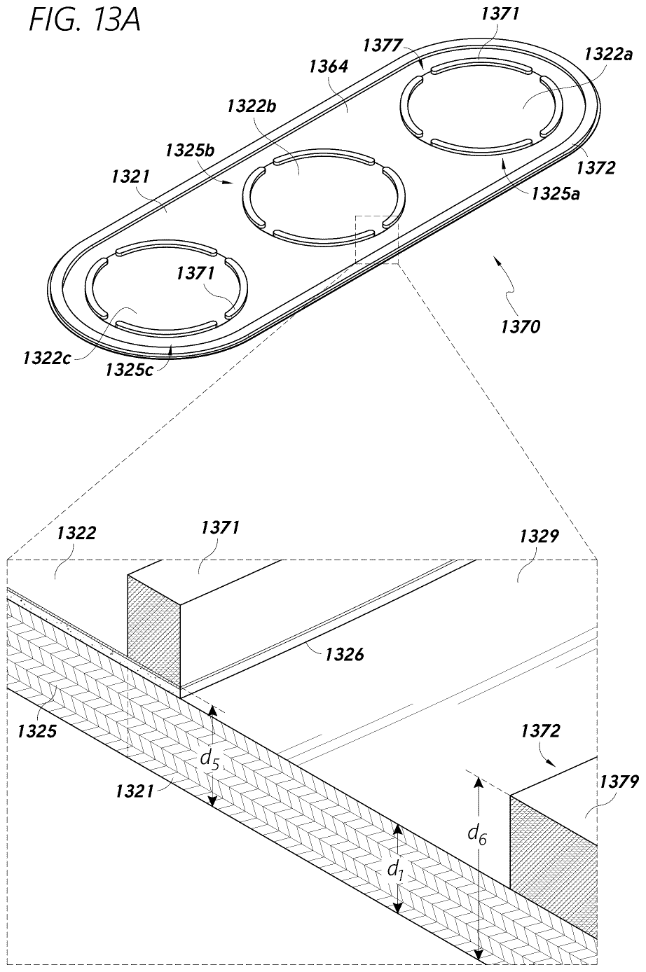

FIG. 12 shows a schematic diagram of a wafer structure 1200 having a plurality of diaphragm structures 1270, or substrate targets for deposition of diaphragm structures, formed therein/thereon in accordance with one or more examples. The wafer 1200 may have formed thereon a plurality of diaphragm molds/substrates and/or thin-film structures, such that a single wafer may be utilized to produce a relatively large number of diaphragm plates/stacks for pressure sensor devices in accordance with aspects of the present disclosure. The reference ‘1270’ in the description below may refer to the shaped substrates of the wafer 1200 onto which nitinol layer/plate is deposited, or may refer to the nitinol layer/plate structure deposited onto the substrate.

The wafer 1200 includes a plurality of diaphragm structures/shapes 1270, wherein each of the shapes 1270 may comprise or be used to produce a separate sensor diaphragm plate. Each of the illustrated plates 1270 may serve as a substrate/mandrel/mold onto which a thin-film diaphragm layer of nitinol metal alloy or similar material may be deposited to form thin-film diaphragm plates comprising, for example, less than 10 μm thickness (e.g., 4-6 μm; around 5 μm) of nitinol or other superelastic material onto which layer(s) of insulator and/or conductor can be applied to produce conformal capacitive electrodes or other similar electronics integrated with superelastic deflectable diaphragms as described in detail herein. The layer(s) of nitinol may have a uniform thickness in both the diaphragm areas 1225, as well as the surrounding structure 1229.

The forms/plates 1270 may be configured with spacing 1201 around one or more portions of the perimeter of the respective diaphragm plate forms 1270, which may facilitate deposition of the nitinol and/or other layers of the diaphragm plates in relatively precise areas and/or shapes, and/or may facilitate singulation of the individual diaphragm plates 1270 after formation thereof to mechanically separate the individual plates from the wafer structure 1200. Physical connectors 1202 may be used to connect the diaphragm plate substrates 1270 to the outer structure 1205 of the wafer 1200, which can serve as a sprue structure. The inside portions 1203 (e.g., the substrates 1270 for the nitinol deposition) of the structure 1200 may be detachable from the wafer 1200, or the deposited layers of material (e.g., nitinol, insulator, electrode) may be removed from the substrate without detachment of the substrate.

The wafer 1200 may be formed of any suitable or desirable material, such as silicone, stainless steel, titanium, nickel, alumina, sapphire, glass, ceramic, or the like. Once the nitinol base diaphragm layer has been deposited on the plate structures 1270, additional layering may be applied to the diaphragm plates using masking and/or other suitable process. For example, on plates 1270 that are intended for use as deflectable diaphragm structures, one or more diaphragm areas 1225 of metal/conductor may be applied to produce capacitive diaphragm plates/electrodes, as described in detail herein.

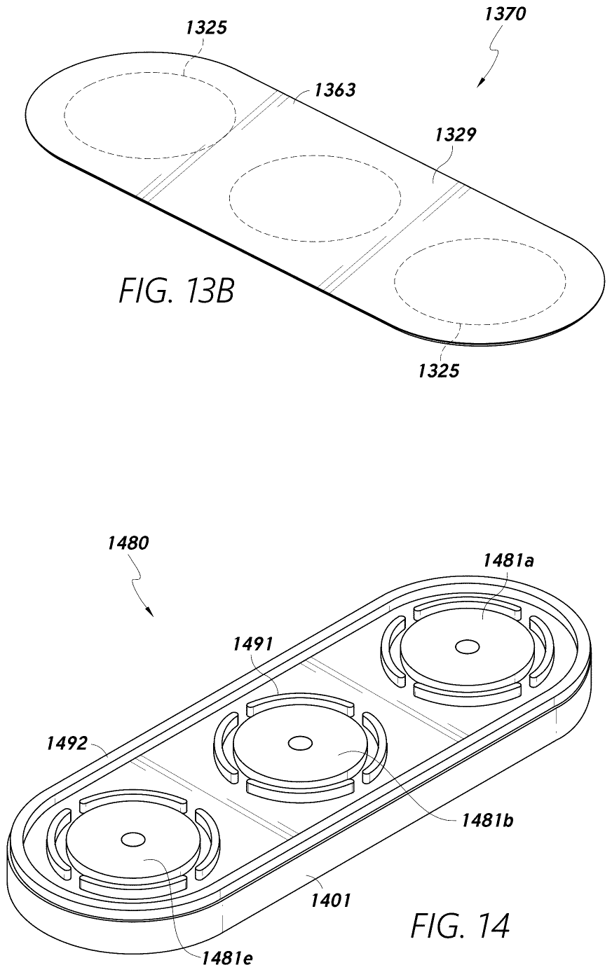

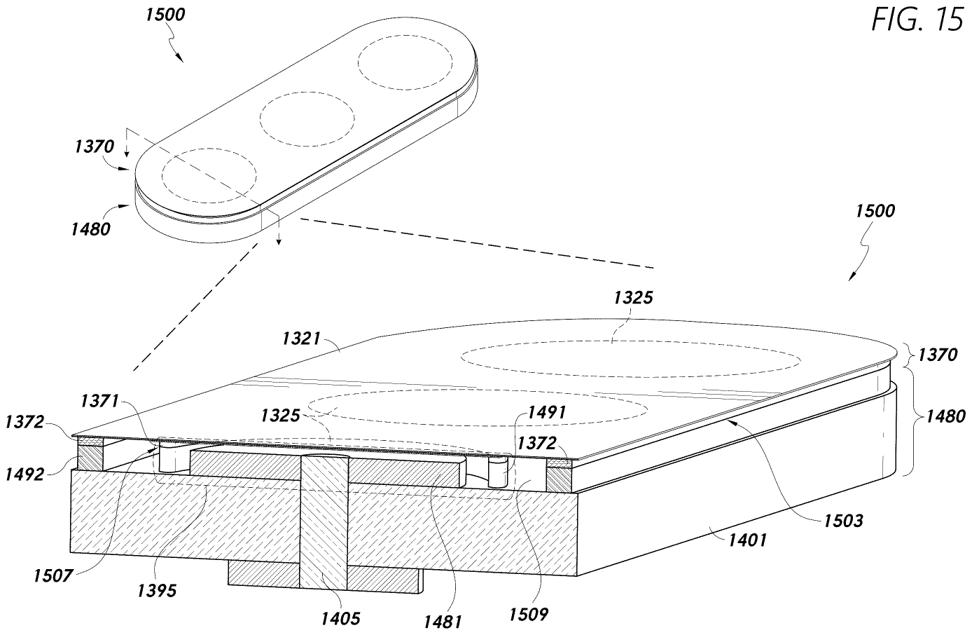

FIGS. 13A and 13B show front and back perspective views of an electrode-integrated sensor diaphragm structure/plate 1370 in accordance with one or more examples. The structure/plate 1370 may provide the structure for one side/plate of a pressure sensing capacitor, wherein certain contacts/connections 1371 of plate 1370 may facilitate electrical connection between electrodes 1322 and a paired electrode base structure (see FIG. 14), wherein the plate 1370 is joined/bonded to counterpart sensor structure in a manner as to provide hermetic sealing thereof. References herein to components that are ‘bonded’ to one another may be understood to be joined in any manner, such as welding, adhesive-bonding, or the like. The contacts 1371 can interface with the electrode(s) 1322 through physical contact therewith. Although the diaphragm plate 1370, as well as certain similar devices/structures disclosed herein, is shown and described as having an oval shape, it should be understood that such devices/structures can have any suitable or desirable shape, such as rectangular, circular, or similar shapes. In some implementations, the active capacitive sensors can occupy the entire surface of the plate 1370, or most of the surface. Alternatively the capacitive sensors can occupy multiple smaller diaphragm areas 1325, as shown. As shown, the diaphragm plate 1370 and/or associated sensor devices/components can have an oval shape resembling the union of two semicircles on opposite sides of a rectangle (referred to in some contexts as an ‘obround’ shape), providing a shape evoking the likeness of a speed skating rink or an athletics track. In some contexts, the shape of the plate/sensor 1370 may be referred to as a “stadium” shape, “disk” shape, or an elongated oval.

The diaphragm plate 1370 includes a sheet/layer of superelastic thin-film nitinol 1321, wherein the nitinol may be deflectable in a dimension normal to the surface of the layer(s) 1321 in one or more areas, such as at least in the areas 1325 corresponding to the capacitor plates 1322. In terms of processing, the nitinol layer 1321, including the diaphragm portion(s) 1325 and the area 1329 outside of the diaphragms 1325, can be deposited on a substrate using physical vapor deposition or other deposition process. The capacitor electrodes/diaphragms 1325 can be distributed along a line or plane, such that they are generally aligned in a linear arrangement.

The insulator/oxide layer(s) 1326 may then be formed or deposited in any suitable or desirable manner on the nitinol 1321. For example, the insulator layer(s) 1326 may be formed only in the areas of the diaphragms 1325. Alternatively, the insulator 1326 may be applied/formed in a manner as to cover substantially the entire inner surface 1364 of the nitinol layer 1321 (i.e., surface shown in FIG. 13A; FIG. 13B shows the outer/back surface 1363 of the nitinol layer 1321). The nitinol layer 1321 may have a thickness d₁ of approximately 5 μm, or any other value less than 10 μm, for example. Although described as ‘thin-film’ diaphragm layers, it should be understood that flexible diaphragm layers disclosed herein may have a thickness of up to 20 μm, or greater in some implementations. The insulator 1326 may be applied to the surface 1364 of the nitinol layer 1321 by masking the areas outside of the diaphragms 1325, such that the insulator/oxide 1326 is formed or deposited solely in the exposed diaphragm areas 1325. The electrode metal 1322 may be applied on the oxide layer(s) 1326, and likewise may be confined to within the areas of the diaphragm(s) 1325. Although three circular diaphragms 1325 are shown, it should be understood that diaphragm plates/structures disclosed herein may have any number, configuration, or shape of diaphragms.

The diaphragm areas 1325 of the nitinol layer 1321 may be identical in form and/or configuration to the areas 1329 outside of the diaphragm(s) 1325, such that the delineation of the diaphragms 1325 may be defined by the area(s) of the insulator 1326 and/or electrode/metal 1322 depositions/layers. In some implementations, the nitinol layer 1321 is materially different in one or more respects in the diaphragm areas 1325 compared to the areas 1329 outside of the diaphragms. For example, the diaphragm portions 1325 may be thinner than the areas 1329 outside of the diaphragm. Additionally or alternatively, certain shape or surface features of the diaphragm areas 1325 may distinguish the diaphragms from the rest of the nitinol sheet/layer. For example, corrugations, protrusions, indentations, impressions, or other features may define an outer perimeter of the diaphragm areas 1325 and/or other areas or features of the diaphragms 1325.

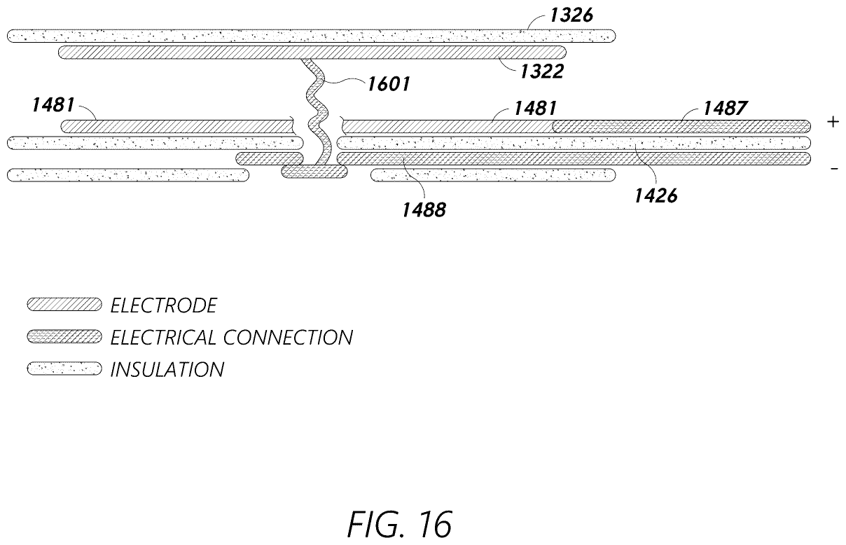

In some implementations, electrical conductor contacts 1371 are applied/formed on the electrode layer(s) 1322, such that the forms 1371 are in electrical contact/communication therewith. For example, the electrical contacts 1371 can be in physical contact with the electrode layer(s) 1322. The contacts 1371 may provide electrical connections between the capacitor plates 1322 and a physically-coupled cathode structure (see FIGS. 14, 15) when the diaphragm plate 1370 is joined (e.g., bonded) thereto, thereby incorporating the diaphragm electrode(s) 1322 in the associated capacitive resonance circuit of the sensor device. The electrical contacts 1371 may have the form of a raised flange, curb, lip, rim, or similar structure, and may generally provide a physical contact surface for electrically interfacing with a counterpart surface 1491 (see FIG. 14) of a base structure.

In some implementations, the electrical contacts 1491 and/or 1371 may comprise electrically-conductive solder, or the like, or other type of material formed as a flange that projects in a dimension normal to a plane of the plate 1370, substrate 1401, and/or device 1500. The contacts 1371 may be formed in any suitable or desirable way, such as through vapor deposition, sputtering, or other application process. In some implementations, due to the thickness of the contact layers 1371, the deflection of the diaphragms 1325 may be confined in some respects to the area within the general perimeter/bounds of the contacts 1371, which may be disposed generally around the perimeter of the electrode(s) 1322. To produce a precise, designed distance between the electrodes 1322 and 1481 in the default, unpressurized/undeflected state (see dimension d₇ in FIG. 10-5), such dimension may be forced by a physical hard stop/contact between the contacts 1491 and 1371, such that the vertical dimension (with respect to the illustrated orientation) of the contact(s) 1491/1371 may be implemented to produce the desired electrode separation d₇. In some implementations, the surface 1364 of the metal layer 1321 is flexible to a degree to allow for the outer/perimeter seal contact 1372 a certain amount of ‘float,’ which may aid in producing a desired hermetic seal.

In some implementations, perimeter gaps 1377 may separate adjacent portions/lengths of the conductors 1371, which may generally run along the perimeter of the electrodes 1322. The gap(s) 1377 may provide a pathway for gas to be removed from the space within the contacts 1371 to allow for vacuum sealing of the chamber 1509. In some examples, the contact(s) 1371 form a continuous perimeter around the electrode(s) 1322 without the presence of circumferential gap(s), which may allow for the implementation of independent and/or isolated capacitors.

The diaphragm plate 1370 may further comprise perimeter-sealing structure 1372, which may be applied to and/or around the perimeter of the plate 1370, such as directly to the thin-film metal (e.g., nitinol) layer 1321. The perimeter structure 1372 may have the form of a raised flange, curb, lip, rim, or similar structure, and may generally provide a sealing contact surface 1379 for sealing against a counterpart surface of a base structure. The sealing flange 1372 can function as a gasket in some respects, and may be incompressible or compressible. The perimeter structure 1372 may project in the direction normal to the surface of the nitinol layer 1321 to provide a structure to offset the capacitor electrode(s) 1322 from structure to which the plate 1370 is bonded. For example, the top surface 1379 of the perimeter structure 1372 may be configured to be bonded to a corresponding surface or feature of a base structure of the sensor device of which the diaphragm plate 1370 is a component. The perimeter seal 1372 may be formed of any suitable or desirable conductive or non-conductive material, and may be applied to the plate 1370 using any suitable or desirable process.

With respect to the back-side surface 1363 of the nitinol layer 1321, as shown in FIG. 13B, the diaphragm areas 1325 may have certain topological surface features associated therewith in accordance with any example disclosed herein, wherein such features may advantageously increase the effective surface area of the diaphragms 1325. For example, such topological features may be formed in the nitinol layer 1321 through the use of a substrate/mandrel surface having such features formed thereon when the nitinol layer 1321 is applied. Alternatively, surface topological features may be formed in the nitinol layer 1321 through additive or subtractive processes (e.g., etching, electroplating, or the like).

FIG. 14 shows a base electrode structure 1480 for the sensor device associated with the diaphragm plate 1370 shown in FIGS. 13A and 13B in accordance with one or more examples. That is, the electrode structure 1480 may serve as a base for the sensor device comprising the physically bonded/combined base structure 1480 and diaphragm plate 1370 (see FIGS. 13A and 13B). FIG. 15 shows the assembled sensor device 1500 including the diaphragm plate 1370 physically coupled/joined to the base structure 1480 in accordance with one or more examples. In some implementations, the diaphragm plate 1370 is welded to the base structure 1480.

The sensor base 1480 can have formed thereon capacitive electrodes 1481, which may each be paired with a corresponding one of the capacitive electrodes 1322 of the diaphragm plate 1370, such that when the plate 1370 is coupled with the base 1480, a variable capacitance, based at least in part on the deflection state of the diaphragm 1325, is present and measurable between the plates 1322, 1481. The electrode(s) 1481, which may have a fixed, non-deflecting attachment/structure, can combine with the deflectable electrode(s) 1322 to form one or more variable capacitors having capacitance that varies in accordance with the deflection state of the electrode(s) 1322. The static nature of the base electrodes (e.g., cathodes) 1481 can be provided by the coupling and/or integration thereof with a rigid, or semi-rigid, substrate structure 1401. Any of the electrodes 1322, 1481 may have an elliptical (e.g., circular) shape, as shown.

The base 1480 may further comprise certain electrical contacts 1491 configured to contact/bond to the corresponding electrical contacts 1371 of the diaphragm plate 1370. That is, the contacts 1491 of the base 1480 may be electrically isolated from the capacitor plates 1481, but electrically coupled to the electrodes 1322 via the contacts 1371, which may be bonded together at coupling interfaces 1507, as shown in FIG. 15, when the diaphragm plate 1370 is bonded to the base structure 1480. While the contacts 1491 may be configured in such a way that they do not directly contact the capacitor plates 1481, through various electrical interconnections, both the capacitor plates 1481 and electrical contacts 1491 can ultimately be connected in the same capacitive residence circuit to allow for the measurement of capacitance between the plates 1322, 1481. The static/fixed capacitor electrodes 1481 may be axially aligned and/or centered with the dynamic capacitor electrodes 1322 when the pieces 1370, 1480 are bonded/coupled together. The electrical contacts 1491 may have the form of a raised flange, curb, lip, rim, or similar structure, and may generally provide a contact surface for electrically interfacing with a counterpart surface 1371 of the diaphragm plate 1370. In some examples, any of the electrical contacts 1371, 1491 are formed as elongated, curved contacts, as shown.

An area of physical contact and sealing between the diaphragm plate 1370 and the base plate 1480 may be via perimeter projections 1492. The dimensions of the electrical contact flanges 1491, 1372 may be designed and set to known distances to offset the capacitive electrodes 1322 and 1481 a desired distance. For example, the opposing flange contacts 1491 and 1371 can be in physical contact to create both the electrical connection between the plates 1370, 1480, as well as defining the precision of the offset of the electrodes 1481, 1322. The contact flanges 1492, 1372 can provide the perimeter seal for the hermetic sealing of the device 1500. In some implementations, the contact flanges 1492, 1372 may have some flexibility to accommodate the relatively complicated construction of the base plate 1401. The perimeter structure 1492 of the base 1480 may advantageously span the entire perimeter of the base substrate 1401, such that, when brought into tight bonding contact with the perimeter structured 1372 of the diaphragm plate 1370, the connection interface 1503 between such perimeter structures can provide a hermetic seal protecting the internal cavity 1509 from the external environment. The internal cavity 1509 can be filled with air or other inert gas, or may be vacuum sealed. In examples in which the cavity 1509 comprises a vacuum, the sensor can provide an absolute pressure sensor. Alternatively, some examples include the presence of a gas in the 1509 cavity/volume, which may be used as a relative pressure sensor implementation. Where a relative pressure sensor is implemented, it may be desirable to implement a tuned reservoir. For example, gaps in the electrode electrical interconnects 1491, 1371 can allow for a relatively large reservoir in the body of the device 1500 beyond the diaphragms 1325, or through a port into another volume.

The substrate 1401 of the base structure 1480 (e.g., cathode structure) can comprise any suitable material, whether rigid or flexible. For example, the substrate 1401 can comprise and/or have associated therewith a printed circuit board and/or a dedicated integrated circuit that contains certain circuitry configured to handle the capacitance signal of the capacitor(s) 1395. Electrical contacts with the capacitor plates 1322, 1481 may be implemented using any suitable or desirable connectivity interconnect design configured to electrically couple the top 1322 and bottom 1481 capacitor electrodes to the relevant electrical circuitry. The base substrate 1401 may include certain electrical connections configured to facilitate proper electrical coupling of the respective capacitor plates 1322, 1481. For example, certain vias or other connections may connect through at least a portion of the thickness of the substrate 1401, such as the via connection 1405 shown, wherein the electrical connections of the sensor 1500 may be implemented within/on the substrate 1401, and/or in/on other areas or structures of the sensor device 1500.

Each of the paired diaphragm plate 1322 and base 1481 capacitive electrodes may combine to form a separate capacitor 1395 of the sensor circuit(s); the sensor may have any number of capacitors 1395, including fewer or more than the illustrated three. In some implementations, the deflection of the diaphragms 1325 are independently measured to provide separate sensor signals, providing additional levels of sensitivity. For example, the different capacitors 1395 may be tuned to have different mmHg/F curves allowing for accounting for (e.g., zeroing-out of) tissue growth that may accumulate disproportionately on one capacitor diaphragm 1325 versus another.

In the example of FIGS. 13-15, the three capacitors 1395 can work in unison to serve as a single capacitor sensor, or they can be separated by certain electrical contacts/connections to make three separate capacitors. The capacitor electrodes 1322 and/or the capacitor electrodes 1481 may comprise gold in some examples. During operation, such as when the sensor 1500 is implanted in a cavity of the human body, the electrodes/plates 1481 may generally remain stationary, even as external pressure conditions fluctuate, whereas the diaphragm electrodes/plates 1322 can dynamically deflect as pressure conditions external to the device 1500 change. The device 1500 can provide a capsule configured to house the active electrical circuitry of the device, which may comprise certain control circuitry configured to transduce changes in capacitance indicated by the deflection state of the diaphragm(s) 1325 and electrode(s) 1322 to electrical signals that can be interpreted, either locally or remotely, to determine a present pressure condition in the environment in which the device is implanted.

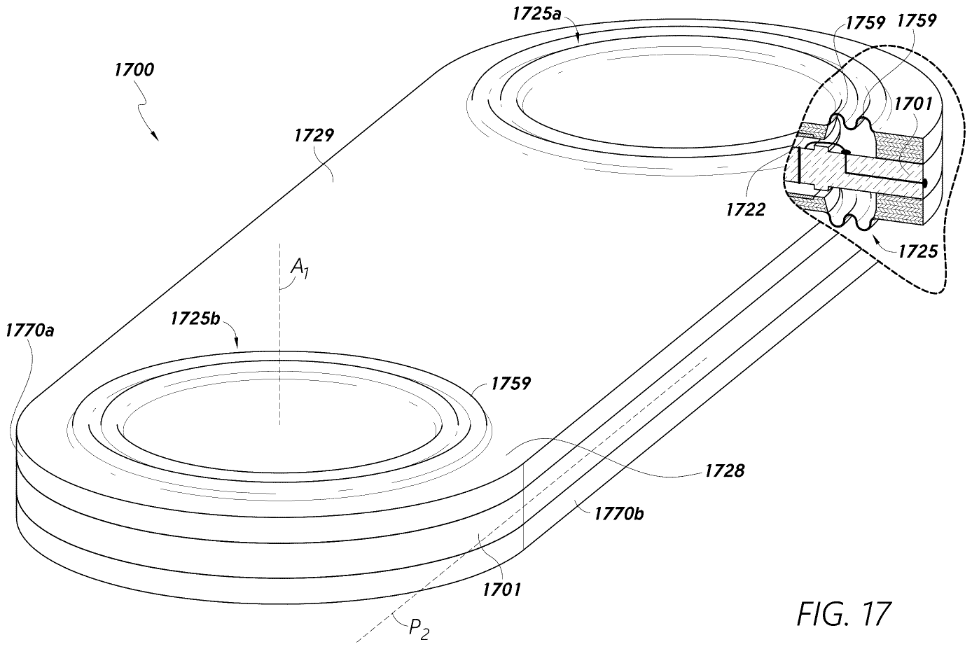

FIG. 16 shows a schematic side view demonstrating example electrical connectivity of components of the capacitive sensor device 1500 as described above. As demonstrated schematically in the image of FIG. 16, the diaphragm electrode 1322 may be electrically coupled via some form of electrical connector(s) 1601 to electrical connector 1488 ultimately providing opposite polarity to the electrical connection 1487 coupled to the base capacitor plate 1481. As described above, such connections to the electrode plates 1322, 1481, may be implemented at least partially within/on the base substrate 1401, and such connections may have certain insulation adjacent thereto to prevent shunting between respective electrodes. The base electrode 1481 may be formed on an insulator/oxide layer 1426 in some implementations. The deflecting electrode 1322 may be coupled to a connector 1601 configured to connect the electrode to the electrical circuit.

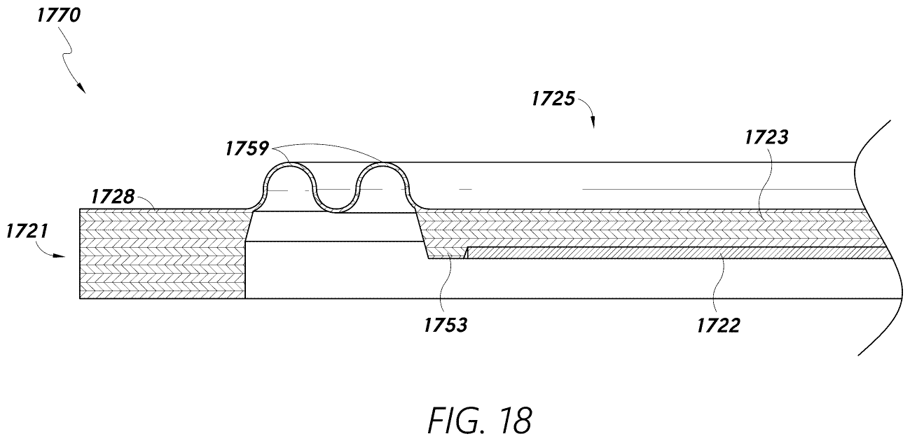

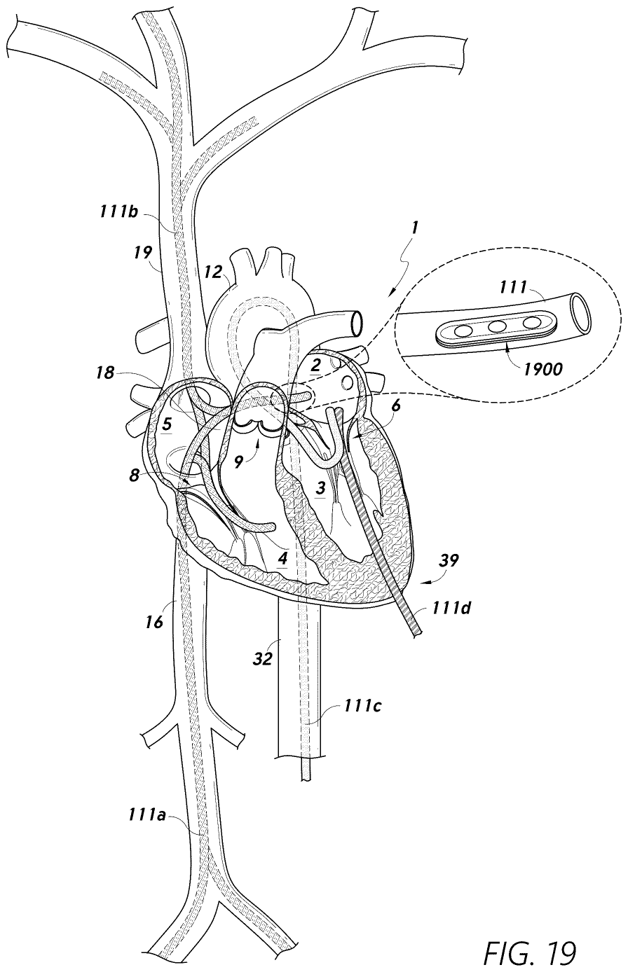

FIG. 17 shows a sensor 1700 having one or more corrugated diaphragms 1725 in accordance with one or more examples. FIG. 18 shows a cross-sectional side view of one of the corrugated diaphragm(s) 1725 of the sensor 1700, showing a conformal capacitive electrode on an underside thereof in accordance with one or more examples. For clarity, the cross-sectional side view of FIG. 18 shows only a single diaphragm plate component of the dual-sided device 1700 of FIG. 17, which may correspond to either of the diaphragm plates 1770a, 1770b of the device 1700.

In order to provide increased deflection sensitivity, the diaphragm(s) 1725 (or any diaphragm disclosed herein) may comprise one or more corrugations 1759. For example, such corrugations 1759 may comprise ring-shaped ridges and/or grooves, which may be concentric with the axis A1 of the diaphragm(s) 1725. Such corrugations 1759 may provide a sufficiently large linear range for the diaphragm(s) 1725 and improved sensitivity. Corrugations may further provide for relatively greater deflection and/or accurate spring rates for the diaphragm(s) 1725 and/or extend the cycle life of the diaphragm(s) 1725 by reducing mechanical stresses in one or more areas of the diaphragm, depending on the particular corrugation design. The corrugations 1759 may be produced by cold-pressing the diaphragm nitinol layer into the corrugated shape, or by any other means. In some implementations, corrugations may be constructed during the physical vapor deposition process by adding shape to the substrate during processing. For example, the corrugation(s) may be formed by shaping the substrate/mandrel (e.g., silicon wafer, glass, etc.) onto which the physical vapor deposition material is deposited. The corrugations may be co-axial with the axis A1 of the diaphragm(s) 1725, as shown. The outer surface 1729 of the nitinol layer(s) 1770 protect the device 1700 from the external environment when implanted.

The corrugated diaphragm 1725 may be implemented in a manner such that the nitinol diaphragm 1725 as formed may have varying thicknesses in different areas thereof. For example, the outer area 1728 of the nitinol layer 1721 outside of the diaphragm 1725 may have a relatively greater thickness compared to the thickness of the nitinol layer in the central diaphragm area 1723 or in the corrugations 1759 (which may or may not have different thickness). The greater thickness of the outer area 1728, which may be formed of multiple layers of nitinol and/or other structural material, can provide a perimeter sealing structure configured to hold the electrode 1722 at a set distance in an un-deflected state thereof relative to a corresponding electrode associated with the base 1701 (see FIG. 17). The central portion 1723 of the diaphragm, which is positioned concentrically within the corrugation(s) 1759, may have a greater thickness than the nitinol in the area of the corrugation 1759, though the thickness of the diaphragm area 1723 may be less than the offsetting/sealing structure 1728. Such relative difference in thicknesses can be visualized with reference to the cross-sectional side image of FIG. 18.

The electrode 1722 may be deposited on an inside surface of the inner diaphragm area 1723. In some implementations, the inner surface of the diaphragm area 1723 may have a recess formed therein, such as through the etching-out of a central area thereof, and/or the building-up of the perimeter area 1753, such that the electrode 1722 is deposited within the recess formed in the inside surface of the diaphragm 1723. That is, with respect to corrugated diaphragm examples disclosed herein of electrode-integrated deflectable nitinol diaphragms, the electrode portions thereof may be confined to an area within the corrugations, such that the electrode is not formed and/or integrated with the corrugation portions themselves.

Although the example sensor 1700 of FIG. 17 shows two diaphragms 1725a, 1725b, it should be understood that corrugated diaphragms may be implemented in connection with any of the examples disclosed herein, and in connection with examples comprising any number and/or configuration of diaphragms.

The example sensor 1700 of FIG. 17 and FIG. 18 further represents an example of a dual-sided sensor device. Although certain examples are illustrated and described herein in the context of only describing a single side of the sensor device having a deflectable diaphragm plate associated therewith that is integrated with capacitive electrode(s), it should be understood that any of the examples disclosed herein may be implemented as dual-sided sensor devices similar in one or more respects to the device 1700 shown in FIG. 17.

Dual-sided sensor devices of the present disclosure may be symmetrical with respect to one or more components thereof across a plane/line P₂ that lies parallel with one or more capacitive electrodes of the device. For example, dual-sided sensor implant devices of the present disclosure may be implemented by including opposite-facing capacitive base electrodes on opposite faces/sides of a static base substrate, such as the base 1701 shown in FIG. 17 (or the base 1401 of FIGS. 14 and 15), wherein first 1770a and second 1770b diaphragm plates may be coupled to the base 1701 on opposite sides thereof, as shown in FIG. 17. The diaphragm plates 1770a, 1770b may be similar or identical in some or all respects.

### Vascular/Cardiac Access for Sensor Implantation

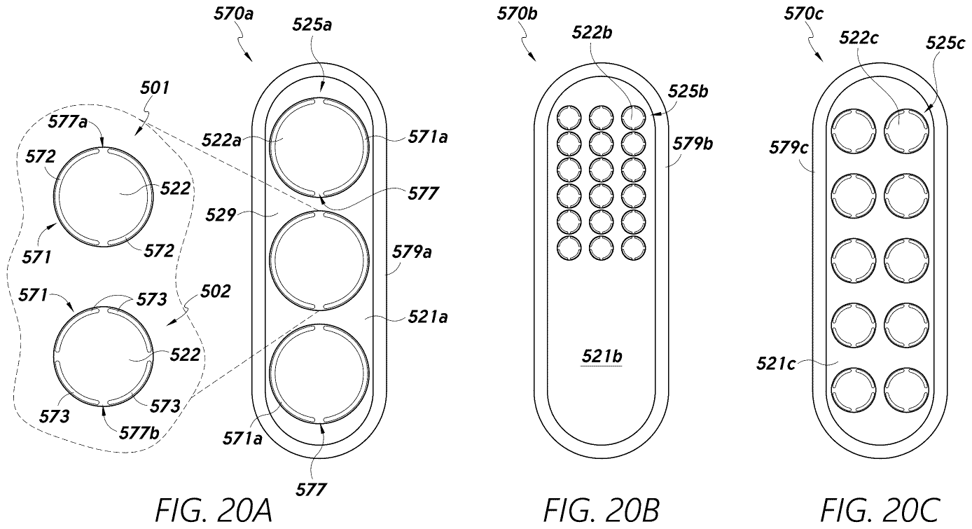

Packaged sensor implant devices in accordance with one or more examples of the present disclosure may be advanced to the relevant target chamber or vessel of the heart and/or vasculature using any suitable or desirable procedure. For example, although access to various chambers/vessels of the heart is illustrated and described in connection with certain examples as being via the right atrium and/or inferior vena cavae, such as through a transfemoral or other transcatheter procedure, other access paths/methods may be implemented in accordance with examples of the present disclosure, as described/shown in connection with FIG. 19. For example, FIG. 19 illustrates various access paths 111 through which access to the chambers of the heart may be achieved using a delivery system. Access to the left atrium or ventricle may be made using transseptal access, which may be made through the inferior vena cava 29 or superior vena cava 19, as respectively shown, and from the right atrium 5, through the septal wall (not shown) and into the left atrium 2. For transaortic access 111c, a delivery catheter may be passed through the descending aorta 32, aortic arch 12, ascending aorta, and aortic valve 7, and into the left atrium 2 through the mitral valve 6. For transapical access 111d, access may be made directly through the apex 39 of the heart into the left ventricle 3, and into the left atrium 2 through the mitral valve 6. Other access paths are also possible beyond those shown in FIG. 19. The various transcatheter delivery systems and paths shown may involve transporting a sensor implant device 1900 within a shaft/lumen of such instrumentation and deploying the device 1900 from the delivery system at the target anatomical site.

### Electrode Configurations

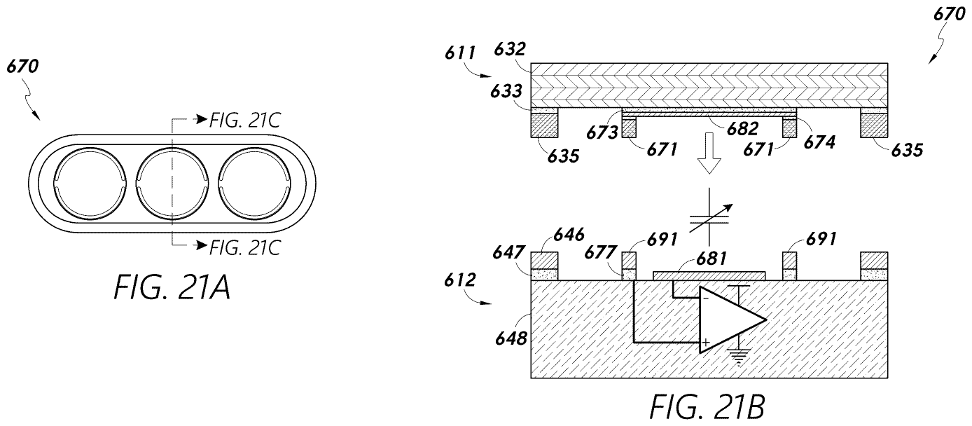

FIGS. 20A, 20B, and 20C show plan views of example electrode-integrated sensor diaphragm/lid structure designs 570. The diaphragm lid structures 570 can be formed at least partially of vapor-deposited nitinol and/or other superelastic material in accordance with one or more examples of the present disclosure. The design of FIG. 20A includes three diaphragms 525a and/or three electrodes 522a (e.g., anodes) in a spaced linear alignment. The electrodes 522a can work in unison to serve as a single electrode or they can have separate electrical contacts to relevant circuitry to serve as three separate electrodes. The electrodes 522a can have respective electrical contacts 571a associated therewith, which may comprise gold, gold-tin, or other electrically conductive material, wherein the contacts 571a are each in contact with a respective one of the three electrodes 522a. The contacts 571a electrically connect the respective electrode 522a (e.g., anode) to a corresponding electrode (e.g., cathode) of a sensor base, thereby forming a capacitance stack of a sensor as described herein.

The sensor diaphragm structures 570 of FIGS. 20A-20C can include perimeter seals 579, which may comprise nitinol, gold-tin, or other material, which provides a hermetic seal that maintains an internal sensor cavity, as disclosed in detail herein, which can be filled with air, inert gas, or a vacuum. In some implementations, the perimeter seal 579 comprises a gold-tin alloy (AuSn), which allows for electrical contact onto the vapor-deposited thin-film nitinol 521 and also provides the hermetic seal. AuSn can be beneficial due to its relatively high eutectic temperature compared to the transformation temperature of the neighboring nitinol 521.

The diaphragm structures 570 (e.g., lids, plates, etc.) can be configured to be joined/bonded to counterpart sensor base structure using a bonding agent, such as gold (Au) or gold-tin (AuSn) alloy layer(s), in a manner as to provide hermetic sealing thereof. References herein to components that are ‘bonded’ to one another may be understood to be joined in any manner, such as welding, adhesive-bonding, or the like. The contacts 571, which may be formed at least in part of gold (Au) or AuSn, can interface with sensor base electrode(s) through direct physical contact or a routing connection through trace(s) on the nitinol diaphragm substrate/layer 521. Although the diaphragm structures 570 are shown and described as having an oval shape, it should be understood that such devices/structures can have any suitable or desirable shape, such as rectangular, circular, or similar shapes.

The diaphragm plates 570 include a sheet/layer of superelastic thin-film nitinol 521, wherein the nitinol may be deflectable in a dimension normal to the surface of the layer(s) 521 in one or more areas, such as at least in the deflectable diaphragm areas 525 corresponding to the capacitor electrodes 522. Insulator layer(s) may be formed in the areas of the diaphragms 525. The nitinol layers 521 may have a thickness of approximately 5 μm, or any other value less than 10 μm, for example. The electrode metals 522 may be applied on dielectric/oxide layer(s) and confined to within the areas of the diaphragm(s) 525. In some implementations, the nitinol layers 521 are materially different in one or more respects in the diaphragm areas 525 compared to the areas 529 outside of the diaphragms. For example, the diaphragm portions 525 may be thinner than the areas 529 outside of the diaphragms. Additionally or alternatively, certain shape or surface features of the diaphragm areas 525 may distinguish the diaphragms from the rest of the nitinol sheet/layer. For example, corrugations, protrusions, indentations, impressions, or other features may define an outer perimeter of the diaphragm areas 525 and/or other areas or features of the diaphragms 525.

The electrical contacts 571 can be in physical contact with the respective electrode layer(s) 522. The contacts 571 may provide electrical connections between the capacitor electrodes 522 and a physically-coupled base sensor structure when the diaphragm plate 570 is joined (e.g., bonded) thereto, thereby incorporating the diaphragm electrode(s) 522 in the associated capacitive resonance circuit of the sensor device. The electrical contacts 571 may have the form of a raised flange, curb, lip, rim, or similar structure, and may generally provide a physical contact surface for electrically interfacing with a counterpart contact of a sensor base structure. The contacts 571 may be formed in any suitable or desirable way, such as through vapor deposition, sputtering, or other application process.

In some implementations, the electrode contacts 571 are formed with perimeter gaps 577, which separate adjacent portions/lengths of the conductors 571 running along the perimeter of the electrodes 522. The gap(s) 577 may provide a pathway for gas to be removed from the space within the contacts 571 and/or to allow for vacuum sealing of the space between the sensor electrodes.

As illustrated by the alternative contact configurations 501, 502 shown in connection with the image of FIG. 20A, the electrode contacts 571 may be implemented with any desirable number of segments. For example, the contact configuration 501 shows an example implementation in which two contact segments 572 are formed on perimeter of the electrode 522, such that two gaps 577a separate the different segments 572. As an example alternative, the configuration 502 includes four contact segments 573 formed on the electrode 522, wherein four gaps 577b around the perimeter of the electrode 522 separate the different contact segments 573. In some examples, the contact(s) 571 form a continuous perimeter around the electrode(s) 522 without the presence of circumferential gap(s), which may allow for the implementation of independent and/or isolated capacitors.

Although three circular diaphragms 525a are shown in FIG. 20A, it should be understood that diaphragm plates/structures disclosed herein may have any number, configuration, or shape of diaphragms. For example, FIGS. 20B and 20C show sensor diaphragm plates 570b, 570c that have different numbers and arrangements of electrodes 522b, 522c and/or diaphragms 525b, 525c, respectively. As shown, electrodes/diaphragms may be arranged in one or more columns and/or rows, with any desirable number and spacing therebetween.

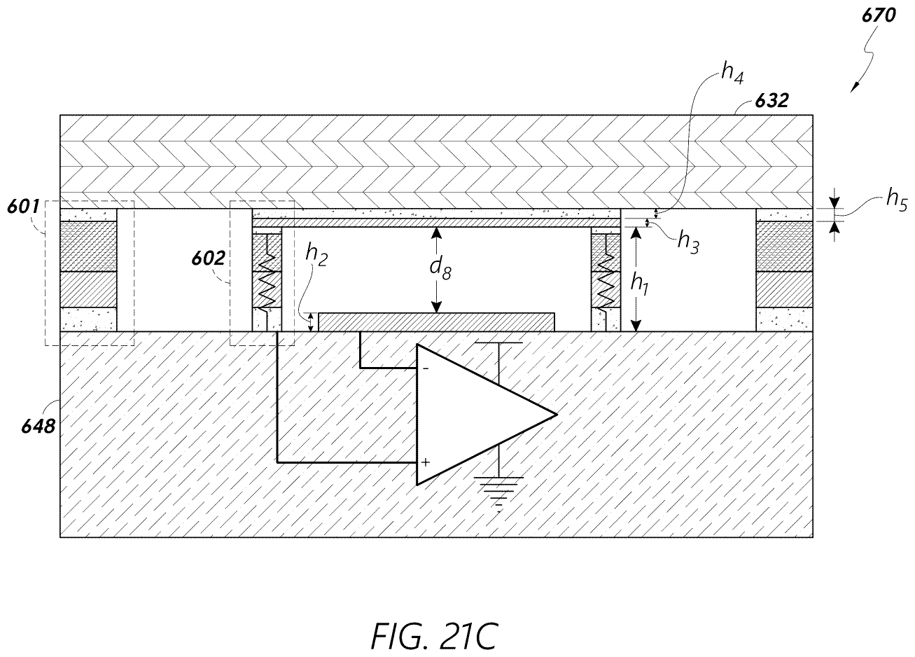

FIG. 21A shows a plan view of a thin-film diaphragm sensor device 670 in accordance with one or more examples. FIGS. 21B and 21C show side exploded and bonded/assembled cross-sectional views, respectively, of example portions of the thin-film diaphragm sensor device 670 shown in FIG. 21A.

FIGS. 21B and 21C includes aspects relating to bond(s) 601, 602 between a diaphragm plate/lid component 611 and a sensor base component 612 that provide hermetic sealing and electrical connection between a nitinol substrate 632 of the diaphragm plate 611 and a non-nitinol substrate 648 of the sensor base 612. Either or both of the substrates 632, 648 may have integrated therewith one or more conductive pathways, passive electronics, and/or active electronics that are electrically connected across the bond(s) 602 in accordance with one or more examples. For example, the electrode bond(s) 602 (and/or perimeter sealing bond(s) 601) can comprise material(s) that provide the capability of electrically connecting functional thin-film nitinol 632 onto a passive (e.g., a printed circuit board (PCB), flex cable, or micro-electro-mechanical system (MEMS) substrate) or an active (e.g., application-specific integrated circuit (ASIC), microprocessor, field-programmable gate array (FPGA), system-on-chip (SOC), microcontroller, radio-frequency identification (RFID) chip, active MEMS, optoelectronic device, etc.) substrate 648.

FIG. 21B shows the thin-film nitinol substrate 632, which may have a passive capacitive electrode 682, or other passive or active electrical element(s), formed thereon. For example, the electrode 682 may comprise a layer of gold (Au) or other conductor applied to the nitinol 632 through physical vapor deposition or other process, as described in detail above. An intermediate layer 673 of high-k dielectric, such as titanium oxide or other suitable oxide, may be interposed between the gold layer 682 and the nitinol 632. Furthermore, form(s) 671 comprising gold-tin (AuSn), Au, or other conductor may be applied onto the electrode 682 to provide electrical contacts to the electrode 682. The nitinol substrate 632 may further include bond tracks 635 outside of the area of the electrode 682 to provide a hermetic bond for the device/structure 630, wherein the bond tracks 635 can be configured to provide a hermetic seal 601 around the electrode 682, such that the assembled device 630 may be suitable for implantation and chronic maintenance within a human body, for example. The hermetic bond forms 635 may be seeded by a seed material 633, such as titanium or other suitable metal/oxide.

The base substrate 648 may comprise any suitable material in/on which electrical elements may be integrated. In some implementations, the base substrate 648 includes bond projections/tracks 646, which may be bonded to the opposite-facing bonds 635 of the diaphragm plate 611 to form the hermetic seals 601. The substrate 648 may further include an electrode or other passive or active circuitry 681. The electrode 681 may serve as a counter electrode to the capacitive electrode 682 associated with the nitinol substrate 632. Therefore, when combined as shown in FIG. 21C, the electrodes 682, 681 may operate as plates of a capacitor, which may be utilized in a pressure-sensing application or other application. Examples of such sensor devices are described in detail above.

The base substrate 648 may further include electrical contacts 691, which may comprise gold or other conductor metal, wherein such contacts 691 are configured to bond to the electrical contacts 671 associated with the diaphragm plate 601. The assembled configuration shown in FIG. 21C shows the formed electrical internal bonds 602 and hermetic perimeter bonds 601, which may connect any number/types of transducers integrated directly onto the thin-film nitinol substrate 632 to the electronics/circuit associated with the corresponding active or passive substrate 648.

In some implementations, the same bond may be formed on the inside 602 and outside 601 of the device 630 in terms of material composition and/or arrangement. The diagram of FIG. 21C represents the electrical connection of the bond 602 by the illustrated resistor icon. Generally, the electrode bond 602 may provide low enough electrical resistance to accommodate use in sensor applications and other applications as described herein.

The bond forms 635 may be implemented as flip-chip bumps/forms to allow for flip-chip combination as demonstrated by the diagrams of FIGS. 21B and 21C. Therefore, according to aspects of the present disclosure, sensor electronics integrated onto a thin-film nitinol substrates (e.g., multiple capacitive electrodes, multiple piezoresistors) can be connected to individual bond forms, wherein joints to such bond forms can be created using traditional flip-chip bonding technologies to bond the nitinol to a flex cable or other substrate.

The cross-sectional views of FIGS. 21B and 21C demonstrate aspects of how a deterministic distance between electrodes (e.g., anode and cathode) of a thin-film diaphragm sensor device in accordance with the present disclosure can be set during sensor assembly/fabrication. Setting the deterministic distance between the electrodes 681, 682 can be primarily done through structure formation on the sensor base substrate 612 rather than on the diaphragm plate 611.

The distance d₈ between the electrodes 681, 682 can generally be set by controlling the difference between the height h₂ of the base electrode 618 (e.g., cathode) and the electrode bond height h₁, which may include one or more of the diaphragm electrode contact 671, the base contact 691, and/or any oxide or seed layers (e.g., layer 377 and/or any present seed layer 674 between the diaphragm contact 671 and electrode 682). Both heights h₁, h₂ may advantageously be repeatable and reproducible using microfabrication technology. For example, the electrode height h₂ can be set by metal sputtering deposition, which may advantageously have a resolution of tens of nanometers. The electrode bond height h₁ can be set by an assembly process using a flip-chip bonder capable of dialing an offset distance between the anode and the cathode during bonding with resolution down to, for example, the hundreds of nanometers. Hard-stop features can be utilized to further control/set the bond height h₁. In some implementations, the thin-film nitinol layer 632 comprises three-dimensional surface features on one or more areas thereof, which provide for a substantial increase in effective surface area of the diaphragm(s). The sensor 670 can advantageously be implemented with the electrodes 681, 682 relatively close to one another without touching to provide for high sensitivity conduction for a given induced pressure.

The electrode distance d₈ can advantageously be on the order of micrometers, typically in the range of several micrometers to several tens of micrometers. While other conductors, such as gold, may be used, it should be understood that gold-tin can provide certain advantages when used as a seed layer to create a hermetic and electrical contact. Furthermore, although the diaphragm plate bonds 635 are shown as additive metal bumps/forms, it should be understood that such features may be formed from protrusions emanating from and built-in/integrated with the thin-film nitinol layer 632.

In fabricating the sensor device 670, the diaphragm plate 611 may be precisely aligned and placed on top of the base structure 612, which may have a similar layer stack-up, or different, forming a capacitor and a hermetic seal. This stack creates a pressure sensitive sensor capable of sustaining large deflections while allowing for large changes in capacitance. The change in capacitance of the capacitor(s) can be read out using passive resonant circuitry, such as a resonant circuit composed of an inductor and capacitor, or active circuitry configured to convert changes in capacitance into digital signals for processing and transmission.

### Additional Description of Examples

Provided below is a list of examples, each of which may include aspects of any of the other examples disclosed herein. Furthermore, aspects of any example described above may be implemented in any of the numbered examples provided below.

Example 1: An implantable sensor device comprising a deflectable diaphragm layer comprising vapor-deposited thin-film metal, a first capacitive electrode conformally formed on a first side of the deflectable diaphragm layer, and a second capacitive electrode coupled to a rigid substrate, the second capacitive electrode and the first capacitive electrode forming a variable capacitor.

Example 2: The implantable sensor device of any example herein, in particular example 1, further comprising a first dielectric layer disposed between the deflectable diaphragm layer and a first side of the first capacitive electrode.

Example 3: The implantable sensor device of any example herein, in particular example 2, wherein the first dielectric layer electrically insulates the first capacitive electrode from the deflectable diaphragm layer.

Example 4: The implantable sensor device of any example herein, in particular example 2, further comprising a second dielectric layer disposed on a second side of the first capacitive electrode.

Example 5: The implantable sensor device of any example herein, in particular example 1, further comprising one or more electrical contacts projecting from the first capacitive electrode, the one or more electrical contacts physically contacting the first capacitive electrode.

Example 6: The implantable sensor device of any example herein, in particular example 5, wherein the first capacitive electrode is elliptical in shape, and the one or more electrical contacts are elongated contacts disposed along a perimeter of the first capacitive electrode.

Example 7: The implantable sensor device of any example herein, in particular example 1, wherein the deflectable diaphragm layer and the first capacitive electrode form a stack having a thickness of less than 10 μm.

Example 8: The implantable sensor device of any example herein, in particular example 1, wherein the deflectable diaphragm layer comprises protrusions associated with one or more sides thereof.

Example 9: The implantable sensor device of any example herein, in particular example 8, wherein the protrusions are formed using etching, masking, or electroplating.

Example 10: The implantable sensor device of any example herein, in particular example 8, wherein the protrusions are corrugations.

Example 11: The implantable sensor device of any example herein, in particular example 1, wherein the first capacitive electrode comprises protrusions associated with one or more sides thereof.

Example 12: The implantable sensor device of any example herein, in particular example 11, wherein the protrusions are formed using etching, masking, or electroplating.

Example 13: An implantable sensor device comprising a first deflectable diaphragm plate comprising a thin-film metal layer and one or more first capacitor electrodes conformally formed on the metal layer. The implantable sensor device further comprises a base structure bonded to the first diaphragm plate to form a sealed cavity, the base structure comprising a rigid substrate and one or more second capacitor electrodes secured to the rigid substrate.

Example 14: The implantable sensor device of any example herein, in particular example 13, wherein the first diaphragm plate further comprises one or more dielectric layers disposed between the metal layer and the one or more first capacitor electrodes.

Example 15: The implantable sensor device of any example herein, in particular example 13, wherein the first diaphragm plate further comprises a first sealing flange projecting from the metal layer and running along a perimeter of the metal layer to provide a sealing contact surface, and a first electrical contact flange projecting from at least one of the one or more first capacitor electrodes.

Example 16: The implantable sensor device of any example herein, in particular example 15, wherein the base structure further comprises a second electrical contact flange projecting from the rigid substrate, the second electrical contact flange being spaced from the one or more second capacitor electrodes.

Example 17: The implantable sensor device of any example herein, in particular example 16, wherein the base structure further comprises a second sealing flange projecting from the rigid substrate around a perimeter of the rigid substrate, the second sealing flange having a surface forming a seal against the sealing contact surface of the first sealing flange.

Example 18: The implantable sensor device of any example herein, in particular example 13, wherein the first diaphragm plate and the base structure have a stadium shape.

Example 19: The implantable sensor device of any example herein, in particular example 13, wherein the one or more first capacitor electrodes comprises a first plurality of capacitor electrodes distributed along a line, and the one or more second capacitor electrodes comprises a second plurality of capacitor electrodes distributed along the line and respectively centered with the one or more first capacitor electrodes.

Example 20: The implantable sensor device of any example herein, in particular example 19, wherein the first plurality of capacitor electrodes and the second plurality of capacitor electrodes form a plurality of capacitors that have a combined capacitance indicative of a pressure level external to the implantable sensor device.

Example 21: The implantable sensor device of any example herein, in particular example 13, further comprising a second deflectable diaphragm plate bonded to an opposite side of the base structure as the first deflectable diaphragm plate.

Example 22: A method of manufacturing an implantable sensor device, the method comprising depositing a layer of thin-film metal on a substrate using a physical vapor deposition process, and depositing a conformal layer of electrical conductor on a stack including the layer of thin-film metal.

Example 23: The method of any example herein, in particular example 22, further comprising, after said depositing the layer of thin-film metal and before said depositing the layer of electrical conductor, forming a first dielectric layer on a surface of the layer of thin-film metal, wherein the layer of electrical conductor is deposited on the first dielectric layer.

Example 24: The method of any example herein, in particular example 23, further comprising forming a second dielectric layer on the layer of electrical conductor.

Example 25: The method of any example herein, in particular example 22, further comprising forming an electrical contact flange on the layer of electrical conductor.

Example 26: The method of any example herein, in particular example 22, further comprising forming surface projections on a surface of the layer of thin-film metal.

Example 27: The method of any example herein, in particular example 22, further comprising forming surface projections on the layer of electrical conductor.

Example 28: The method of any example herein, in particular example 22, further comprising bonding a plate structure including at least the layer of thin-film metal and the layer of electrical conductor to a base structure comprising a capacitive electrode to form a sealed cavity including a space between the layer of electrical conductor and the capacitive electrode.

Example 29: The method of any example herein, in particular example 28, further comprising forming a sealing flange in or on the layer of thin-film metal, wherein said bonding the plate structure to the base structure involves joining a contact surface of the sealing flange to the base structure.

Example 30: The method of any example herein, in particular example 22, further comprising forming one or more corrugations in a portion of the layer of thin-film metal.

Example 31: The method of any example herein, in particular example 30, wherein the one or more corrugations are co-axial with the layer of electrical conductor.

Example 32: A sensor device comprising a deflectable diaphragm layer comprising a flexible material deposited using a gas vapor deposition process, a first capacitive electrode conformally formed on a first side of the deflectable diaphragm layer, and a second capacitive electrode, the second capacitive electrode and the first capacitive electrode forming a variable capacitor.

Example 33: A sensor device comprising a deflectable diaphragm layer comprising a flexible material deposited using a gas vapor deposition process, and a conductive electrode formed conformally formed on a first side of the deflectable diaphragm layer.

Example 34: The sensor device of any example herein, in particular example 33, wherein the conductive electrode is a piezoresistor.

Example 35: The sensor device of any example herein, in particular example 33, wherein the conductive electrode is a capacitor plate.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. An implantable sensor device comprising:
a deflectable diaphragm layer comprising vapor-deposited thin-film metal;
a first capacitive electrode conformally formed on a first side of the deflectable diaphragm layer; and
a second capacitive electrode coupled to a rigid substrate, the second capacitive electrode and the first capacitive electrode forming a variable capacitor.

2. The implantable sensor device of claim 1, further comprising a first dielectric layer disposed between the deflectable diaphragm layer and a first side of the first capacitive electrode.

3. The implantable sensor device of claim 2, wherein the first dielectric layer electrically insulates the first capacitive electrode from the deflectable diaphragm layer.

4. The implantable sensor device of claim 2, further comprising a second dielectric layer disposed on a second side of the first capacitive electrode.

5. The implantable sensor device of claim 1, further comprising one or more electrical contacts projecting from the first capacitive electrode, the one or more electrical contacts physically contacting the first capacitive electrode.

6. The implantable sensor device of claim 5, wherein:
the first capacitive electrode is elliptical in shape; and
the one or more electrical contacts are elongated contacts disposed along a perimeter of the first capacitive electrode.

7. The implantable sensor device of claim 1, wherein the deflectable diaphragm layer and the first capacitive electrode form a stack having a thickness of less than 10 μm.

8. The implantable sensor device of claim 1, wherein the deflectable diaphragm layer comprises protrusions associated with one or more sides thereof.

9. The implantable sensor device of claim 8, wherein the protrusions are formed using etching, masking, or electroplating.

10. The implantable sensor device of claim 8, wherein the protrusions are corrugations.

11. The implantable sensor device of claim 1, wherein the first capacitive electrode comprises protrusions associated with one or more sides thereof.

12. The implantable sensor device of claim 11, wherein the protrusions are formed using etching, masking, or electroplating.

13. An implantable sensor device comprising:
a housing configured for implantation within a patient, the housing including a diaphragm plate and a base structure hermetically coupled together;
a deflectable diaphragm integrated with the diaphragm plate, the deflectable diaphragm being formed of vapor-deposited thin-film superelastic metal;
an electrically insulating layer conformally formed on an underside of the deflectable diaphragm;
a first conductive electrode conformally vapor-deposited on the electrically insulating layer and electrically isolated from the deflectable diaphragm;
a second conductive electrode supported by the base structure and positioned opposite the first conductive electrode to form a variable capacitor;
sensor circuitry electrically coupled to the first conductive electrode and the second conductive electrode and configured to detect changes in capacitance of the variable capacitor corresponding to deformation of the deflectable diaphragm; and
a telemetry component coupled to the sensor circuitry and configured to wirelessly communicate pressure-related signals generated by the sensor circuitry to an external device.

14. The implantable sensor device of claim 13, wherein the first conductive electrode includes a plurality of electrical contact pads formed along a perimeter region on a base-structure-facing side of first conductive electrode, the plurality of electrical contact pads being formed by physical vapor deposition and providing electrical coupling between the first conductive electrode and the sensor circuitry.

15. The implantable sensor device of claim 14, wherein:
the base structure includes a plurality of raised conductive contacts disposed around, and spaced from, a perimeter of the second conductive electrode; and
the plurality of electrical contact pads of the first conductive electrode are configured to physically and electrically engage the plurality of raised conductive contacts to establish electrical communication between the first conductive electrode and the sensor circuitry.

16. The implantable sensor device of claim 14, wherein the plurality of electrical contact pads comprise a plurality of circumferentially distributed arc-shaped contact segments defining a discontinuous perimeter band, adjacent arc-shaped contact segments being separated by gaps that provide fluid communication between a volume beneath the first conductive electrode and a region outside a perimeter boundary of the deflectable diaphragm within a sealed chamber of the implantable sensor device.

17. The implantable sensor device of claim 16, wherein the diaphragm plate includes a vapor-deposited hermetic sealing band formed on the diaphragm plate, the hermetic sealing band comprising a continuous, closed-loop raised metallization region that circumscribes the first conductive electrode.

18. The implantable sensor device of claim 13, wherein the deflectable diaphragm includes one or more annular corrugations formed in a peripheral region of the deflectable diaphragm, the one or more annular corrugations defining an inner diaphragm region that supports the first conductive electrode.

19. An implantable sensor device comprising:
a diaphragm plate configured for an implantable sensor housing, the diaphragm plate comprising a vapor-deposited thin-film superelastic metal layer, the diaphragm plate including a plurality of deflectable diaphragm regions, each of the plurality of deflectable diaphragm regions being configured to elastically deform in response to physiological pressure forces;
a plurality of insulation pads conformally formed on a face of the diaphragm plate, each of the plurality of insulation pads corresponding to a respective one of the plurality of deflectable diaphragm regions;
a plurality of conductive diaphragm electrodes, each conformally deposited on a respective one of the plurality of insulation pads using vapor-deposition of conductive metal material;
a plurality of raised electrode contact structures disposed along perimeter regions of respective ones of the plurality of conductive diaphragm electrodes; and
a hermetic sealing band vapor-deposited on the diaphragm plate in a continuous, closed-loop raised metallization structure that runs circumferentially about a perimeter of the diaphragm plate and dimensioned to form a hermetic seal with a corresponding base structure to enclose a sealed sensor chamber.

20. The implantable sensor device of claim 19, wherein each of the plurality of raised electrode contact structures comprises circumferentially distributed arc-shaped contact segments defining a segment of a discontinuous perimeter band.
